(19) United States
(12) Patent Application Publication
(10) Pub. No.: US 2018/0089363 A1
(43) Pub. Date: Mar. 29, 2018

(54) METHOD FOR EXTRACTING LEAD COMPOUND, METHOD FOR SELECTING DRUG DISCOVERY TARGET, DEVICE FOR CREATING SCATTER DIAGRAM, AND DATA VISUALIZATION METHOD AND VISUALIZATION DEVICE

(71) Applicant: ONO PHARMACEUTICAL CO., LTD., Osaka (JP)

(72) Inventors: Masakuni KURONO, Osaka (JP); Hiromu EGASHIRA, Osaka (JP); Jun TAKEUCHI, Osaka (JP)

(73) Assignee: ONO PHARMACEUTICAL CO., LTD., Osaka (JP)

(21) Appl. No.: 15/567,741

(22) PCT Filed: Apr. 21, 2016
(86) PCT No.: PCT/JP2016/062659

§ 371 Date: Oct. 19, 2017

(30) Foreign Application Priority Data
Apr. 22, 2015 (JP) 2015-087915

(51) Int. Cl. G06F 19/16; G06F 19/26

(52) U.S. Cl. CPC G06F 19/16; C12Q 1/485; G06F 19/26

(57) ABSTRACT

A method for extracting a lead compound from a plurality of compounds against a drug discovery target, includes the steps of creating a scatter diagram for a plurality of compounds by disposing symbols representing the compounds according to a plurality of features of the compounds and extracting a lead compound from the compounds represented by the symbols disposed in a predetermined region of the scatter diagram. The locations of the symbols to be disposed on the scatter diagram are determined according to first and second features (for example, selectivity and activity) of the respective compounds, and attributes (for example, color and size) of the symbols are determined according to third and fourth features (for example, molecular weight and ligand efficiency) of the respective compounds.

### TECHNICAL FIELD

The present invention relates to a method for extracting a lead compound, a method for selecting a drug discovery target, and a device for creating a scatter diagram used for these methods. The present invention also relates to a data visualization method, and a visualization device.

### BACKGROUND ART

The success rate of drug development is very low. It is said that only one in 30,591 newly researched drug candidate compounds successfully makes it to the market as a new drug. Acquisition of quality lead compounds is therefore important in improving the success rate, and delivering a new drug to the market in as small a time frame as possible.

A lead compound is a “drug-like” compound that shows activity and a pharmacological effect against a target of drug discovery (hereinafter, also referred to as “drug discovery target”), and that can be used as a starting point of further optimization (lead optimization).

A lead compound rarely becomes a drug by itself. For approval as a drug candidate compound, a lead compound needs to be studied from a wide range of perspectives, including, for example, strength of activity, the selectivity of the main activity against other activities, a pharmacological effect in animal experiments, pharmacokinetics, safety, stability of the active pharmaceutical ingredient, manufacturing cost, and patentability, and all of these requirements need to be satisfied by a lead compound. In order to meet these requirements, a lead compound is commonly used as a starting point for a wide range of synthetic expansion.

In different lead compounds, a compound that can be expected to have high potential for synthetic expansion can be said as a quality lead compound.

A lead compound is selected from compounds (hit compounds) showing activity higher than a certain reference level through compound screening against a drug discovery target. The result of compounds screening is visualized in the form of, for example, a heat map, which can then be used to select a lead compound. In another known method, a two-dimensional scatter diagram is created for activity and selectivity, and a compound having high activity and high selectivity is selected (NPL 1, NPL 2).

The recently developed combinatorial chemistry and high-throughput screening techniques have enabled diversified screening of a wide range of compound libraries in a short time period. The advance in information processing techniques has also enabled computer processing of a large volume of data having several million data points.

A heat map is a convenient display system as long as the relationship between compounds and activity value is viewed in a single map. A drawback, however, is the difficulty in grasping data in a comprehensive fashion, and handling of data becomes a laborious process when the process involves numerous data points. A two-dimensional scatter diagram enables selection of a compound group having high activity and high selectivity. However, it is not possible to determine whether the compound group has good potential for synthetic expansion.

### CITATION LIST

### Patent Literature

PTL 1: JP-A-2015-1943

### Non Patent Literature

NPL 1: High-throughput kinase profiling as a platform for drug discovery, David M. Goldstein, et al., Nature Reviews Drug Discovery, 2008, 7, 391-397
NPL 2: CASE Plots for the Chemotype-Based Activity and Selectivity Analysis: A CASE Study of Cyclooxygenase Inhibitors, Jaime Perez-Villanueva, et al., Chem Biol Drug Des., 2012, 80, 752-762
NPL 3: For Bridging of Creative Drug Discovery Research (Souzouteki Souyaku Kenkyu no Hashiwatashi ni Mukete), National Institute of Biomedical Innovation, Pamphlet (http://www.nibio.go.jp/part/promote/fundamental/pdf/link. pdf)

### SUMMARY OF INVENTION

### Technical Problem

There accordingly is a need for a method for extracting a quality lead compound from numerous data obtained from a wide range of compound libraries, and a method for selecting a drug discovery target having good potential for synthetic expansion.

The present invention is intended to provide a method for extracting or selecting a lead compound and a drug discovery target having good potential for synthetic expansion. The invention is also intended to provide a scatter diagram creating device for creating a scatter diagram used for the method.

### Solution to Problem

The present inventors diligently worked to find a solution to the foregoing problems, and found that a quality lead compound can be selected by creating a four-dimensional scatter diagram that uses the activity, selectivity, molecular weight, and ligand efficiency values obtained by screening. Specifically, a visualization method was found that uses a four-dimensional scatter diagram of numerous data points for the selection of a quality lead compound, and that can be used to comprehensively speculate the possibility of synthetic expansion. The present invention has been completed on the basis of these findings.

With the four-dimensional scatter diagram, it is possible to determine whether a drug candidate compound would be created against the drug discovery target of interest in the future after synthetic expansion even when a quality lead compound cannot be found at the time when the four-dimensional scatter diagram is created.

The four-dimensional scatter diagram also enables determining whether a compound library for a given drug discovery target should be used for synthetic expansion. That is, it is possible to determine the suitability of a compound library against a drug discovery target.

In a first aspect of the present invention, there is provided a method for extracting a lead compound from a plurality of compounds against a drug discovery target. The method includes the steps of: creating a scatter diagram for a plurality of compounds by disposing symbols representing the respective compounds according to a plurality of features of the respective compounds; and extracting a lead compound from the compounds represented by the symbols disposed in a predetermined region of the scatter diagram. A location of the symbol to be disposed on the scatter diagram is determined according to first and second features of the compound, and attributes of the symbol are determined according to third and fourth features of the compound.

In a second aspect of the present invention, there is provided a method for selecting a drug discovery target. The method includes the steps of: creating a scatter diagram for a plurality of compounds against a predetermined molecular target, by disposing symbols representing the respective compounds according to a plurality of features of the respective compounds; and selecting the predetermined molecular target as a drug discovery target according to a distribution of the symbols disposed on the scatter diagram. A location of the symbol to be disposed on the scatter diagram is determined according to first and second features of the compound, and attributes of the symbol are determined according to third and fourth features of the compounds. The compounds are divided into a plurality of groups under a predetermined condition regarding the third feature. In the selecting step, it is determined whether to select the predetermined molecular target as a drug discovery target, according to a direction and an endpoint of change in the distributions of the symbols of the compounds belonging to the respective groups.

In a third aspect of the present invention, there is provided a scatter diagram creating device for creating a scatter diagram that represents features of a plurality of compounds against a predetermined drug discovery target. The device includes: an obtaining unit for obtaining feature information regarding various features of the compound, for a plurality of compounds; and a scatter diagram creating unit for creating a scatter diagram for the plurality of compounds, by disposing symbols representing the compounds according to the obtained feature information, and outputting the scatter diagram.

The scatter diagram creating unit determines the locations of the symbols to be disposed on the scatter diagram according to first and second features of the respective compounds, determines attributes of the symbols according to third and fourth features of the respective compounds, and disposes the symbols representing the compounds on the scatter diagram according to the determined locations and the determined attributes.

In a fourth aspect of the present invention, there is provided a method for visualizing a pattern of a plurality of data having at least first to fourth features. The method includes: determining a location on which a symbol representing each piece of data is to be disposed, according to the first and second features; determining attributes of the symbol representing each piece of data, according to the third and fourth features; and disposing the symbol representing each piece of data on a scatter diagram according to the determined location and the determined attributes.

In a fifth aspect of the present invention, there is provided a device for visualizing a pattern of a plurality of pieces of data having at least first to fourth features. The device includes: an obtaining unit for obtaining feature information regarding features of data, for each piece of data; and a scatter diagram creating unit for creating a scatter diagram according to the feature information obtained for the data.

The scatter diagram creating unit determines the location on which a symbol representing each piece of data is disposed, according to the first and second features, determines attributes of the symbol representing each piece of data, according to the third and fourth features, and disposes, on the scatter diagram, the symbol representing each piece of data according to the determined location and the determined attributes.

In a sixth aspect of the present invention, there is provided a second method for extracting a lead compound from a plurality of compounds against a drug discovery target. The method includes the steps of: creating a scatter diagram for a plurality of compounds by disposing symbols representing the respective compounds according to a plurality of features of the respective compounds; and extracting a lead compound from the compounds represented by the symbols disposed in a predetermined region of the scatter diagram.

Locations of the symbols to be disposed on the scatter diagram are determined according to first and second features of the respective compounds. The first feature is selectivity of the compound against the predetermined drug discovery target. The second feature is activity of the compound against the predetermined drug discovery target. The predetermined region is a region in which the selectivity and the activity are equal to or greater than respective predetermined values. A compound having a ligand efficiency of 0.3 or more is extracted from the compounds represented by the symbols disposed in the predetermined region.

In a seventh aspect of the present invention, there is provided a second method for visualizing a pattern of a plurality of data having at least first to third features. The method includes: determining a location on which a symbol representing each piece of data is disposed, according to the first and second features; disposing the symbol representing each piece of data on a scatter diagram according to the determined location; dividing the plurality of pieces of data into a plurality of groups under a predetermined condition regarding the third feature; and disposing an arrow connecting centers of distributions of the symbols of the data belonging to the respective groups on the scatter diagram.

### Advantageous Effects of Invention

According to the lead compound extraction method of the present invention, a candidate lead compound is extracted from a predetermined region of a scatter diagram, and a quality lead compound having good potential for synthetic expansion can be extracted.

According to the drug discovery target selecting method of the present invention, a predetermined target is selected as a drug discovery target to be used for drug discovery, on the basis of the direction and the end point of a change in the distribution of compound symbols within each group divided with regard to a third feature. In this way, the method enables selecting a drug discovery target having good potential for synthetic expansion.

The scatter diagram creating device of the present invention can provide a scatter diagram that is desirable for the extraction of a lead compound, or for the selection of a drug discovery target. In the scatter diagram, the location of the compound symbol plotted on the scatter diagram is set according to the first and the second feature of the compound, and the attributes (color, size) of the symbol are set according to the third and the fourth feature of the compound. In this way, the four features of the compound can be visually grasped at the same time. The scatter diagram also enables grasping data in a comprehensive fashion, and predicting the possibility of synthetic expansion.

According to the visualization device and the visualization method of the present invention, the four features of data of interest for analysis can be visually recognized at the same time, and the patterns of the analyzed data can be easily grasped.

### BRIEF DESCRIPTION OF DRAWINGS

FIG. 1 is a diagram showing an example of a four-dimensional scatter diagram in which symbols representing a plurality of compounds are plotted against a predetermined drug discovery target according to different features of each compound.

FIGS. 2A and 2B show two-dimensional scatter diagrams representing an existing form of visualization for the activity and selectivity of an inhibitory compound against two kinases (drug discovery targets).

FIGS. 3A and 3B show four-dimensional scatter diagrams for an inhibitory compound against two kinases (drug discovery targets) visualized according to an embodiment of the present invention.

FIGS. 4A and 4B show four-dimensional scatter diagrams in which arrows for predicting the possibility of synthetic expansion are disposed.

FIGS. 5A and 5B represent diagrams in which the arrows for predicting the possibility of synthetic expansion are disposed alone.

FIG. 6 shows diagrams representing four-dimensional scatter diagrams for five kinases (drug discovery targets) displayed side by side.

FIG. 7 shows diagrams in which the arrows for predicting the possibility of synthetic expansion are shown by themselves after being generated from the four-dimensional scatter diagrams for the five kinases (drug discovery targets).

FIG. 8 is a diagram representing the result of an evaluation of several tens of thousands of compounds against target C.

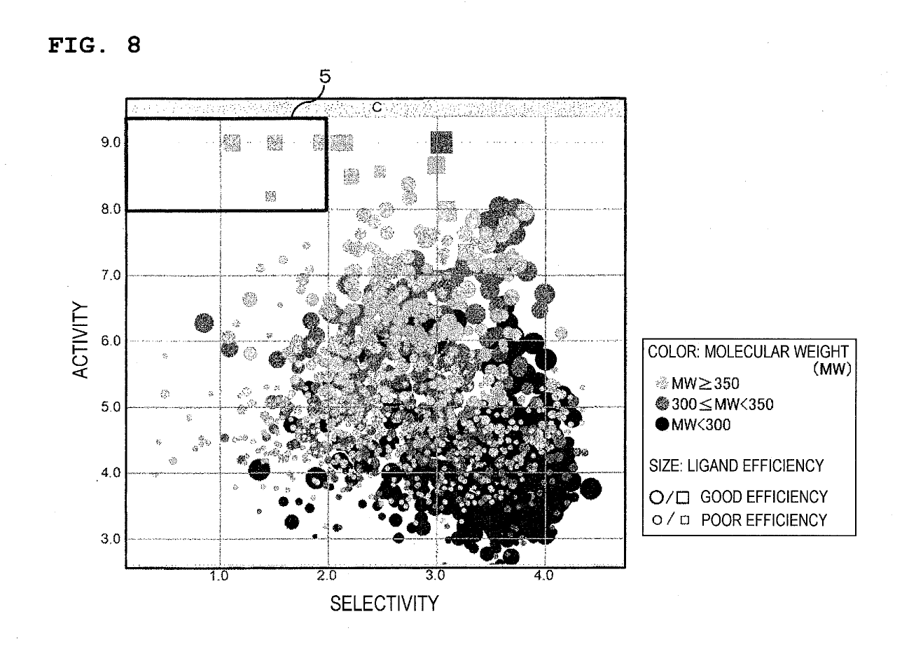

FIG. 9 is a diagram representing a hardware configuration of a four-dimensional scatter diagram creating device.

FIG. 10 is a flowchart representing the four-dimensional scatter diagram display operation of the four-dimensional scatter diagram creating device.

FIGS. 11A and 11B show diagrams describing boxes that represent a first priority region and a second priority region in a high-activity and high-selectivity region.

FIG. 12 is a flowchart representing the process by which the arrow for predicting the possibility of synthetic expansion is generated in the four-dimensional scatter diagram creating device.

FIG. 13 shows a flowchart representing the process for determining a promising drug discovery target.

FIG. 14 is a diagram representing another display example of the arrow for predicting the possibility of synthetic expansion against a plurality of drug discovery targets.

FIG. 15 is a diagram representing yet another example of how the arrow for predicting the possibility of synthetic expansion is displayed against a plurality of drug discovery targets.

FIG. 16 is a diagram representing an example of a four-dimensional scatter diagram for weather data.

FIG. 17 is a diagram representing an example of a four-dimensional scatter diagram for medical data.

### DESCRIPTION OF EMBODIMENTS

Embodiments of the present invention are described below with reference to the accompanying drawings.

As used herein, the term “molecular target” means a functional macromolecule that, within a living organism, is closely associated with the causes of clinical disorders and diseases, and that can be controlled by some means to prevent and/or treat the disease. Specific examples of the molecular target include:

Receptors (for example, cell surface receptors such as ion-channel-coupled receptors, tyrosine kinase-coupled receptors, and G protein-coupled receptors; and nuclear receptors such as retinoic acid receptors, and steroid hormone receptors), enzymes (for example, oxidation-reduction enzymes such as dehydrogenase, reductase, oxidase, oxygenase, and hydroperoxidase; transferases such as methyltransferase, hydroxymethyltransferase, formyltransferase, carboxyltransferase, carbamoyltransferase, amidetransferase, acyltransferase, aminoacyltransferase, glycosyltransferase, aminotransferase, oximinotransferase, phosphotransferase (for example, kinase), nucleotidyltransferase, sulfatransferase, sulfotransferase, and CoA transferase; hydrolases such as protease, esterase, glycosidase, and peptidase; lyases such as aldolase, decarboxylase, dehydratase, and carboxykinase; isomerases such as racemase, epimerase, cis-transisomerase, sugar isomerase, tautomerase, Δ-isomerase, mutase, and cycloisomerase; and ligases such as DNA ligase),

transporter proteins (for example, ion-channels, and ion pumps), and

nucleic acids (for example, micro-RNA, RNA, and DNA).

As used herein, the term “drug discovery target” means a molecular target of interest for drug discovery. The drug discovery target is preferably an enzyme, more preferably a transferase, particularly preferably a kinase. Aside from enzymes, the drug discovery target may be a receptor, or a transporter protein.

As used herein, the term “lead compound” means a compound having activity on the drug discovery target, and whose activity on molecular targets other than the drug discovery target is weaker than the activity on the drug discovery target, and that can become a possible drug compound through chemical modification. It is not necessarily the case that the activity of the lead compound on the drug discovery target is sufficiently strong. Depending on the drug of interest, it may be desirable to use a lead compound that has activity on two or more drug discovery targets.

As used herein, “scatter diagram” is a diagram in which data are plotted in the form of symbols with corresponding quantities, for example, weight and size, against two parameters (features) represented by the vertical and horizontal axes. That is, the data has, for example, a weight and a size against two parameters (features).

### First Embodiment

### 1. Four-Dimensional Scatter Diagram

First, a four-dimensional scatter diagram is described that is used for extraction of a lead compound, or selection of a drug discovery target.

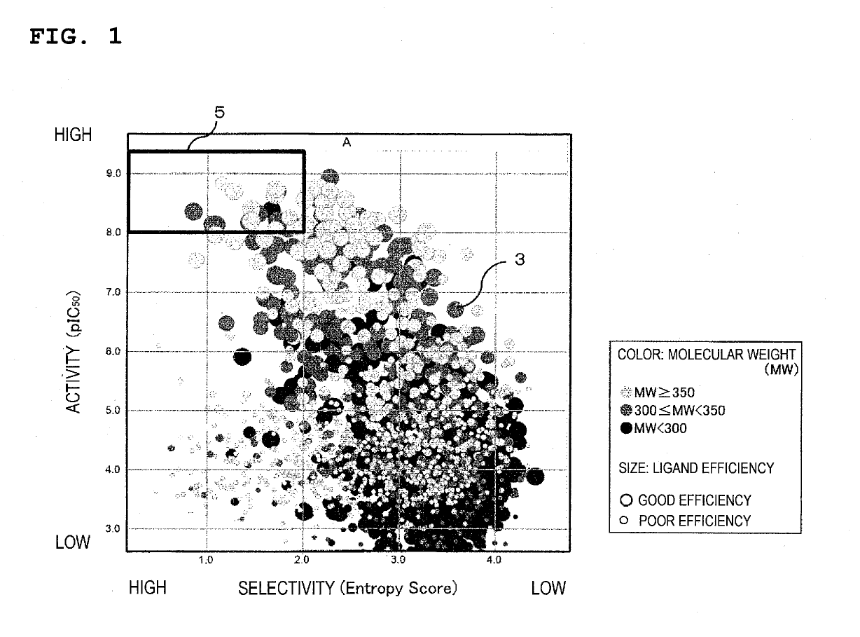

FIG. 1 is a diagram representing an example of the four-dimensional scatter diagram of the present embodiment. The four-dimensional scatter diagram shown in the figure is a scatter diagram plotting a plurality of compounds against a kinase of interest (an example of the drug discovery target or the molecular target) on the basis of four parameters, which include the activity value (for example, pIC₅₀), the selectivity (for example, entropy score), the ligand efficiency, and the molecular weight of the compounds. As shown in the figure, the four-dimensional scatter diagram is created by plotting selectivity on the horizontal axis (X axis) and activity value on the vertical axis (Y axis), and symbols 3 (open circle marks) representing compounds are plotted on the two-dimensional plane of selectivity-activity values. The color and size of the symbol 3 representing a compound are determined by the molecular weight and the ligand efficiency, respectively, of the compound (details will be described later). The four-dimensional scatter diagram enables visually grasping the four features of the compound at the same time, and understanding the data in a comprehensive fashion. This makes it possible to predict the possibility of synthetic expansion.

The following describes the methods for calculating the activity value, the selectivity, and the ligand efficiency used to create the four-dimensional scatter diagram.

### (1) Calculation of Activity Value

Examples of the activity of a lead compound against the drug discovery target include receptor binding activity, receptor control activity, receptor signaling activation activity, receptor signaling inhibition activity, enzyme control activity, enzyme activation activity, enzyme inhibition activity, channel binding activity, channel control activity, channel activation activity, channel inhibition activity, pump binding activity, pump control activity, pump activation activity, pump inhibition activity, and protein-protein interaction inhibitors.

The notation used for activity value is not particularly limited, and the activity value may be represented by, for example, activation rate, inhibition rate, control rate, half maximal effective concentration (EC₅₀) pEC₅₀, half maximal inhibitory concentration (IC₅₀), pIC₅₀, estimated half maximal inhibitory concentration (eIC₅₀) peIC₅₀, 50% lethal concentration (LC₅₀), pLC₅₀, activation constant (Kₐ), pKₐ, inhibition constant (Kᵢ), pKᵢ, dissociation constant (K_{d}) pK_{d}, median effective dose (ED₅₀) pED₅₀, median inhibitory dose (ID₅₀) pID₅₀, median lethal dose (LD₅₀), pLD₅₀, association rate constant (kₒₙ), dissociation rate constant (k_{off}), residence time, free energy (ΔG), enthalpy (ΔH), entropy (ΔS), or melting temperature (Tm). Preferred are activation rate, inhibition rate, half maximal effective concentration, pEC₅₀, half maximal inhibitory concentration, pIC₅₀, activation constant, pKₐ, inhibition constant, pKᵢ, dissociation constant, and pK_{d}. More preferred are half maximal effective concentration, pEC₅₀, half maximal inhibitory concentration, pIC₅₀, activation constant, pKₐ, inhibition constant, pKᵢ, dissociation constant, and pK_{d}. Particularly preferred are half maximal inhibitory concentration (IC₅₀), and pIC₅₀.

As an example, the activity value is represented by half maximal inhibitory concentration IC₅₀ (pIC₅₀) in the present embodiment. The following describes the method of calculation of half maximal inhibitory concentration IC₅₀ (pIC₅₀) for enzyme inhibition activity.

Five milliliters of a 4× concentration test substance solution (several thousand compounds) prepared with an assay buffer (20 mM HEPES, 0.01% Triton X-100, 2 mM DTT, pH 7.5), five milliliters of a 4× concentration substrate/ATP/metal ion (magnesium ions with optional manganese ions; the ion choice depends on the kinase) solution, and ten milliliters of a 2× concentration kinase solution (several hundred different kinases) were mixed in the wells of a 384-well polypropylene plate, and reacted at room temperature for 1 or 5 hours (depending on the kinase). The reaction was quenched by adding 60 mL of Termination Buffer (QuickScout Screening Assist MSA; Carna Biosciences). The substrate peptide and the phosphorylated peptide in the reaction solution were separated, and quantified with the LabChip 3000 system (Caliper Life Science). The kinase reaction was evaluated using the product ratio (P/(P+S)) calculated from the substrate peptide peak height (S), and the phosphorylated peptide peak height (P).

The inhibition rate (%) was calculated from a signal of each well of the tested substance. In the calculation, the average signal of the control well containing all reaction components was given as 0% inhibition, and the average signal of the background well (containing no enzyme) was given as 100% inhibition.

The compound concentration that inhibited the phosphorylation of the substrate by 50% was defined as IC₅₀. The IC₅₀ value was calculated by least squares method by substituting the calculated inhibition rate in the following logistic formula.

Y=Bottom+(Top−Bottom)/(1+10̂(HillSlope×(log IC₅₀−log₁₀(X)))

In the formula, Y is the inhibition rate (%), X is the concentration, Top is the maximum inhibition rate (100 in this experiment), Bottom is the minimum inhibition rate (0 in this experiment), and HillSlope is the slope (1 in this experiment).

When the formula did not satisfy determination coefficient R²>0.5, and Log IC₅₀ maximum error <1, the IC₅₀ value was calculated by using the inhibition rate (%) for the maximum evaluation concentration, as follows.

IC₅₀=100×X/Y−X,

where Y is the inhibition rate (%), and X is the concentration (μM).

When the inhibition rate (%) at the maximum evaluation concentration was 20% or less, that is, when there was no activity, a fixed value was used for the subsequent calculation of the entropy score used as an index of selectivity. In this experiment, the IC₅₀ value was 4,000 μM when the maximum evaluation concentration was 10 μM, and 40,000 μM when the maximum evaluation concentration was 100 μM.

The IC₅₀ value calculated above was used as an activity value after converting it to a pIC₅₀ value, or a molar concentration −log IC₅₀ value.

### (2) Calculation of Selectivity

The selectivity of a lead compound means the activity ratio of the lead compound against the drug discovery target of interest relative to the activity against molecular targets other than the drug discovery target.

The index of the selectivity of a lead compound against the drug discovery target is not particularly limited. Examples include entropy score, selectivity entropy, information entropy, Shannon entropy, selectivity score, selectivity index, Gini coefficient, Gini score, and partition coefficient. Preferred are entropy score, selectivity score, selectivity index, Gini coefficient, and partition coefficient. More preferred are Gini coefficient, and entropy score. Particularly preferred is entropy score.

As an example, entropy score was used as an index of selectivity in the present embodiment. The entropy score was calculated from the calculated IC₅₀ value above, according to BMC Bioinformatics, 2011, 12, 94. Aside from the entropy score, it is possible to use other selectivity indices, including, for example, selectivity score (Nature Biotechnology, 2008, 26, 1, 127), Gini coefficient (J. Med. Chem., 2007, 50, 23, 5773), and partition coefficient (J. Med. Chem., 2010, 53, 11, 4502).

### (3) Calculation of Ligand Efficiency

The ligand efficiency is an evaluation index of a compound, estimating the strength of activity of the molecule by size.

The index of ligand efficiency is not particularly limited. Examples include ligand efficiency, percentage efficiency index, binding efficiency index, surface-binding efficiency index, fit quality score, percent ligand efficiency, group efficiency (GE), and ligand lipophilicity efficiency (LLE). Preferred are ligand efficiency, percentage efficiency index, binding efficiency index, and surface-binding efficiency index. More preferred are ligand efficiency, and percentage efficiency index. Particularly preferred is ligand efficiency.

In the present embodiment, the ligand efficiency was calculated using the calculated IC₅₀ value above, and the number of atoms (heavy atoms) excluding the hydrogens in the compound, according to the literature (Drug Discovery Today, 2005, 10, 987).

The four-dimensional scatter diagram shown in FIG. 1 was created using the four features, specifically, the activity value (pIC₅₀), the selectivity (entropy score), and the ligand efficiency calculated for the drug discovery target in the manner described above, and the molecular weight. Specifically, symbols 3 representing compounds were plotted with the activity value and the selectivity representing the vertical axis (Y axis) and the horizontal axis (X axis), respectively, of the four-dimensional scatter diagram. The symbols 3 were plotted in different colors for different molecular weights. In the example of FIG. 1, the compounds were divided into three groups: a first group with a molecular weight of less than 300, a second group with a molecular weight of 300 or more and less than 350, and a third group with a molecular weight of 350 or more, and the symbols 3 representing the compounds have different colors (for example, red, yellow, and blue) for these groups.

The size of the symbol 3 was varied with the ligand efficiency. In the example of FIG. 1, the symbols 3 have larger sizes for larger ligand efficiency values, and smaller sizes for smaller ligand efficiency values. The symbols 3 were represented by a size larger than a certain size when the ligand efficiency value was larger than a certain value, and by a size smaller than a certain size when the ligand efficiency value was smaller than a certain value.

When pIC₅₀ is used as activity value, the pIC₅₀ of a lead compound is preferably 4 or more, more preferably 5 or more, particularly preferably 6 or more. When the selectivity is entropy score, the entropy score of a lead compound is preferably 4 or less, more preferably 3 or less, particularly preferably 2 or less. The molecular weight of a lead compound is preferably 500 or less, more preferably 400 or less, particularly preferably 350 or less. The ligand efficiency of a lead compound is preferably 0.25 or more, more preferably 0.3 or more, particularly preferably 0.35 or more.

In the four-dimensional scatter diagram shown in FIG. 1, compounds with larger activity values on the vertical axis have stronger activity, and compounds with smaller selectivity values on the horizontal axis have higher selectivity. For extraction of a lead compound, the four-dimensional scatter diagram has a predetermined region with preferably a pIC₅₀ of 6 or more, and an entropy score of 4 or less, more preferably a pIC₅₀ of 7 or more, and an entropy score of 3 or less, particularly preferably a pIC₅₀ of 8 or more, and an entropy score of 2 or less, when pIC₅₀ is used as activity value, and entropy score is used for the evaluation of selectivity. Specifically, a region with an activity of 8 or more, and a selectivity of 2 or less represents a region containing compounds that are particularly desirable as lead compounds. Accordingly, a box representing a high-activity and high-selectivity region 5 is disposed on the four-dimensional scatter diagram. The high-activity and high-selectivity region 5 is a region containing compounds that are more desirable as lead compounds. Compounds that are desirable as lead compounds can be easily recognized by focusing on the compounds contained in the region 5.

As a rule, a lead compound is preferably a high-activity and high-selectivity compound with a lower molecular weight. In the four-dimensional scatter diagram, the symbols have different colors according to the molecular weight, and improved activity and selectivity due to a molecular weight change can be easily recognized. In the four-dimensional scatter diagram, the ligand efficiency is represented by a symbol size that varies with the ligand efficiency value. In this way, an active compound having good efficiency can be grasped in one glance even when it has a small molecular weight. Compounds with larger symbols (open circle marks) are compounds that have efficiently gained activity (see FIG. 1).

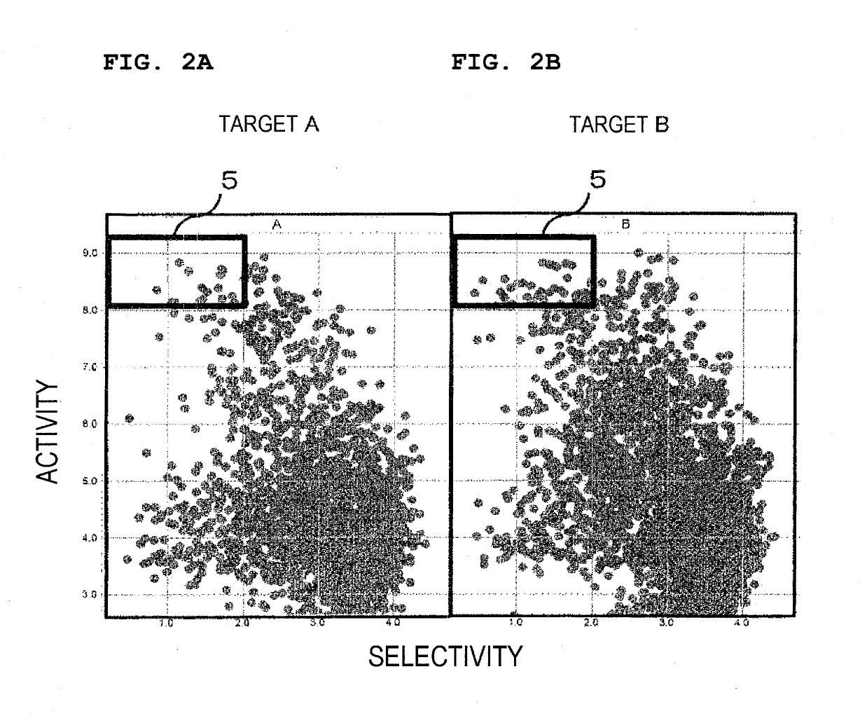

FIGS. 2A and 2B show two-dimensional scatter diagrams representing an existing form of visualization for activity and selectivity against two kinases (drug discovery targets) A and B. For both kinases A and B, compounds are plotted in the high-activity and high-selectively region 5. With the existing form of visualization, it is unclear whether the high-activity and high-selectivity compounds are possible candidate of quality lead compounds.

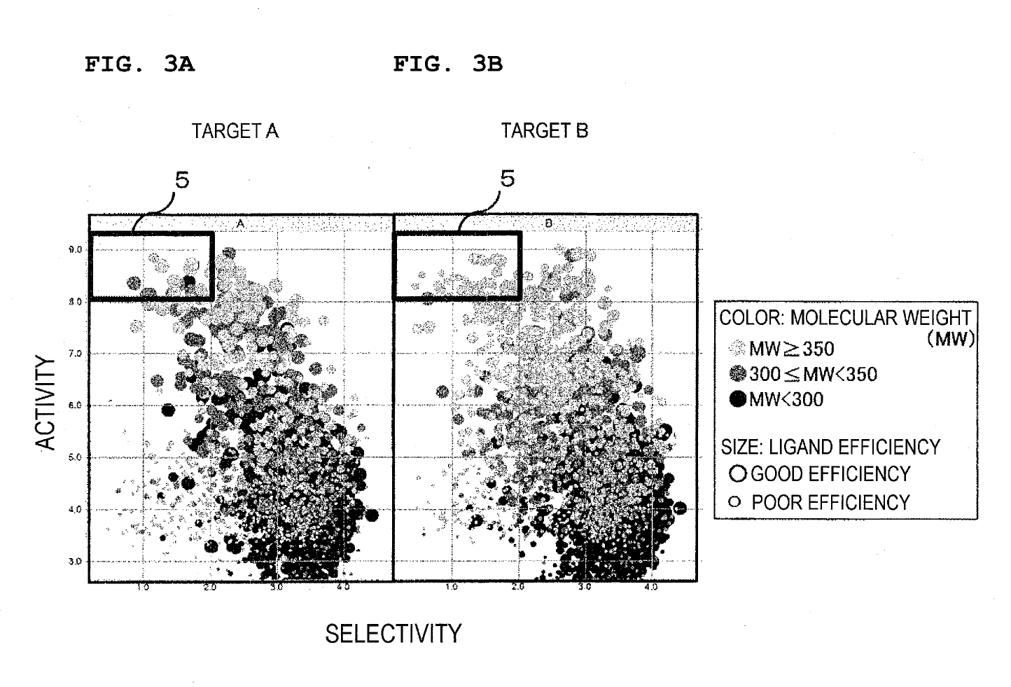

FIGS. 3A and 3B show four-dimensional scatter diagrams of the embodiment of the invention against kinases (drug discovery targets) A and B. With the four-dimensional scatter diagram shown in FIGS. 3A and 3B, it can be understood how the molecular weight, an important factor of a quality lead compound, is distributed, and the ligand efficiency can be recognized in one glance. For example, referring to FIG. 3A, a plurality of compounds having good ligand efficiency, and a molecular weight of less than 300, and a molecular weight of 300 or more and less than 350 is present in the region 5 for kinase A. In contrast, referring to FIG. 3B, most of the compounds in the region 5 for kinase B are compounds having poor ligand efficiency, and a molecular weight of 350 or more. Compounds with poor ligand efficiency are not suited as lead compounds even when they have high activity and high selectivity. That is, it can be seen that a more desirable quality lead compound can be obtained for kinase A than for kinase B.

### 2. Lead Compound Extraction Method

The high-activity and high-selectivity region 5 in the four-dimensional scatter diagram is a region containing compounds that are more desirable as lead compounds. A compound is therefore extracted from the group of compounds contained in the region 5. This enables extraction of a compound desirable as a lead compound. A compound satisfying predetermined molecular weight and/or ligand efficiency conditions also may be selected from the group of compounds contained in the high-activity and high-selectivity region 5. The predetermined molecular weight condition may be, for example, a molecular weight equal to or less than a predetermined value. The predetermined ligand efficiency condition may be, for example, a ligand efficiency equal to or greater than a predetermined value. For example, a compound having a ligand efficiency of 0.3 or more may be extracted as a lead compound from the compounds contained in the high-activity and high-selectivity region 5. A compound having a molecular weight of 350 or less, and a ligand efficiency of 0.3 or more may also be extracted as a lead compound from the compounds contained in the high-activity and high-selectivity region 5.

### 3. Displaying Arrow for Prediction of Possibility of Synthetic Expansion

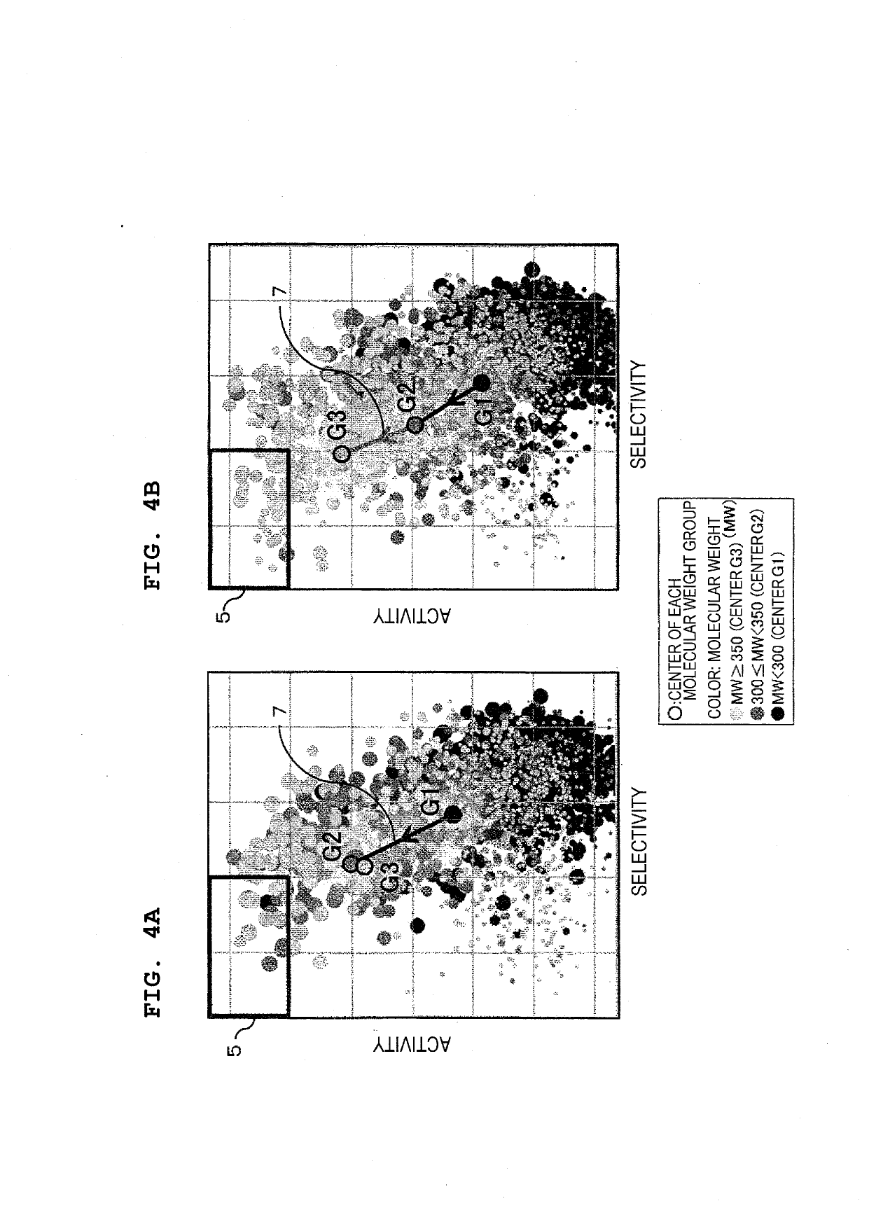

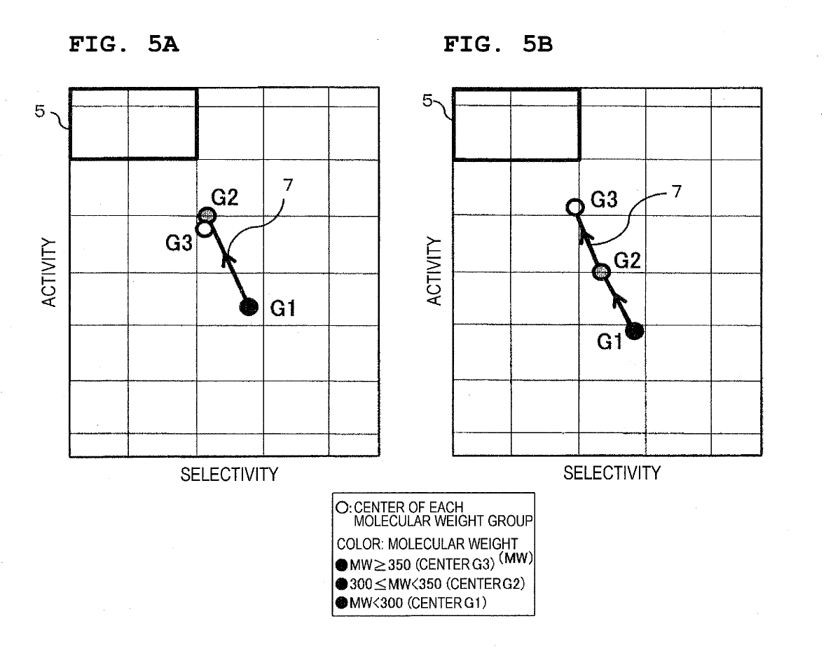

FIGS. 4A and 4B show four-dimensional scatter diagrams in which an arrow 7 for predicting the possibility of synthetic expansion is disposed, in addition to the symbols. FIGS. 5A and 5B show diagrams showing the arrow 7 for predicting the possibility of synthetic expansion, centers G1, G2, and G3 of compound distributions, and a preferred region for the center of a compound distribution, excluding the symbols plotted in the diagrams shown in FIGS. 4A and 4B. By referring to the arrow 7 disposed in the four-dimensional scatter diagram, it is possible to predict the possibility of synthetic expansion from a lead compound for the kinase of interest represented in the four-dimensional scatter diagram (i.e., a molecular target as a candidate drug discovery target), and to determine whether the kinase of interest (molecular target) is suited as a drug discovery target.

The arrow 7 was determined by excluding compound data that had an inhibition rate of 20% or less at the maximum evaluation concentration. For each kinase, compound data was used that had above-average values for activity value (pIC₅₀), selectivity, and ligand efficiency data in each molecular weight group. Instead of using data with above-average values as in this example, it is possible to use an arbitrary number of higher-ranked data.

For each kinase, the centers G1, G2, and G3 of compound distributions on the selectivity-activity two-dimensional plane were calculated for each of the three molecular weight groups, and connected with an arrow 7 between groups of the adjacent molecular weight ranges, as shown in FIGS. 4 and 5. Specifically, the arrow 7 connected the center G1 to G2, and the center G2 to G3. The arrow 7 indicates the direction of change of the center of the distribution from a smaller to a larger molecular weight (i.e., the direction of change of the distribution). The center G1 indicates the starting point of a distribution change, and the center G3 indicates the endpoint of a distribution change. The centers G1, G2, and G3 represent the centers of the distributions on the selectivity-activity two-dimensional plane for the first to third groups that are based on the molecular weight. Specifically, the centers G1, G2, and G3 are determined for the feature values of activity and selectivity, as follows.

Gx=(X1+X2+ . . . +Xn)/n (1)

In the formula, Xn is the activity value (Y-coordinate value) or the selectivity value (X-coordinate value), Gx is the center (x=1 to 3) of the feature value, and n is the number of compounds belonging to each group based on the molecular weight.

Alternatively, the activity value data, and the selectivity data may be weighted with the ligand efficiency data using standardized values of activity, selectivity, and ligand efficiency, and the weighted arrow 7 may be determined for each kinase from the centers of activity value and selectivity calculated for each molecular weight group.

Sx=(Xi−Xmin)/(Xmax−Xmin) (2)

In the formula, Xi is the activity value (Y-coordinate value) or the selectivity value (X-coordinate value) (i=1 to n), Sx is the feature value after standardization, Xmin is the minimum value, and Xmax is the maximum value.

Wz=(Wi−Wmin)/(Wmax−Wmin) (3)

In the formula, Wi is the ligand efficiency value (i=1 to n), Wz is the feature value after standardization, Wmin is the minimum value, and Wmax is the maximum value.

G′x={(S1×W1)+(S2×W2)+ . . . +(Sn×Wn)}/ΣWi (4)

In the formula, G′x is the center (x=1 to 3) of the weighted feature value.

### 4. Drug Discovery Target Selecting Method

Whether a given molecular target is suited as a drug discovery target is determined from the locations of the centers G1, G2, and G3 determined for the molecular target, and the direction of the arrow between the centers G1 and G2, and between the centers G2 and G3. Specifically, a molecular target is determined as being suited as a drug discovery target when the molecular target satisfies the following condition A, and at least one of the conditions B1, B2, and B3.

### Condition A

The arrow between the centers G1 and G2 (the arrow from center G1 to center G2) is directed toward the region (toward the upper left of the scatter diagram; hereinafter, the region will also be referred to as “high-activity and high-selectivity region 5”).

### Condition B1

The center G2 is contained in the high-activity and high-selectivity region 5.

### Condition B2

The arrow between the centers G2 and G3 (the arrow from center G2 to center G3) is directed toward the region (toward the upper left of the scatter diagram), and the center G3 representing the end point of change of the distribution is contained in the high-activity and high-selectivity region 5.

### Condition B3

The arrow between the centers G2 and G3 is directed toward the region (toward the upper left of the scatter diagram), and the center G3 representing the end point of change of the distribution is contained in a predetermined range of activity value (pIC₅₀ of 5 or more).

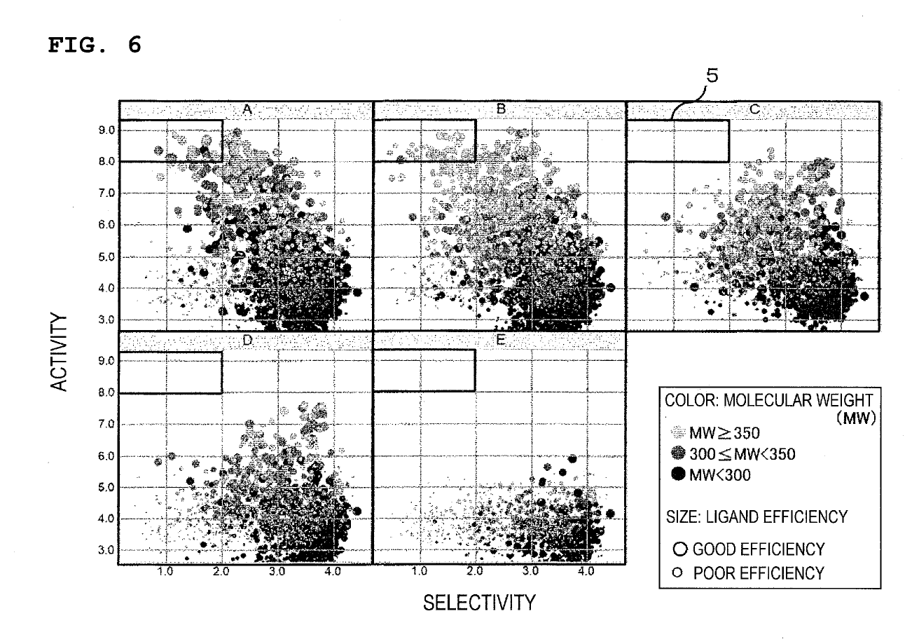

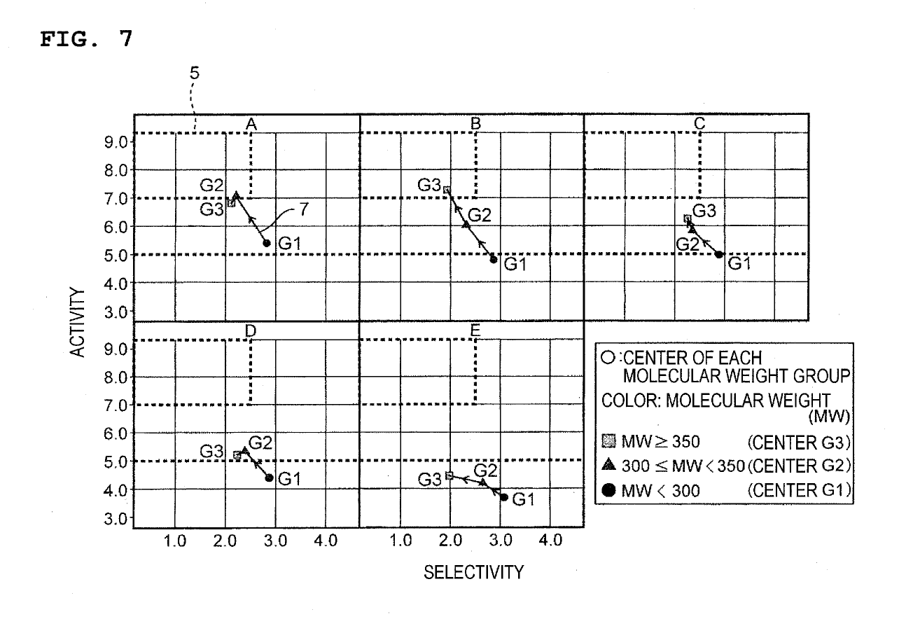

FIG. 6 shows exemplary four-dimensional scatter diagrams for five different molecular targets (kinases) A to E. FIG. represents diagrams created from the four-dimensional scatter diagrams for the molecular targets A to E, showing the arrow 7 for predicting the possibility of synthetic expansion, the centers G1, G2, and G3 of compound distributions, a preferred region for the centers of compound distribution, and the predetermined range of activity value. In FIG. 7, the high-activity and high-selectivity region 5 is a region with an activity (pIC₅₀) >7.0, and a selectivity (entropy score) <2.5, and the predetermined range of activity value is a pIC₅₀ of 5 or more.

### Molecular Target A

The center G2 of the group of compounds with a molecular weight of 300 or more and less than 350 is plotted closer to the upper left side than the center G1 of the group of compounds with a molecular weight of less than 300 (condition A), and the center G3 is contained in the high-activity and high-selectivity region 5 (activity (pIC₅₀) >7.0, selectivity (entropy score)<2.5) (condition B1). That is, the molecular target A satisfies condition A and condition B1, and can be determined as a promising drug discovery target.

### Molecular Target B

The center G2, and the center G3 of the group of compounds with a molecular weight of 350 or more are plotted closer to the upper left side than the center G1 (condition A), and the center G2 is contained in the high-activity and high-selectivity region 5 (condition B2). That is, the molecular target B satisfies condition A and condition B2, and can be determined as a promising drug discovery target.

### Molecular Target C

The center G2, and the center G3 are plotted closer to the upper left side than the center C1 (condition A). However, the center G2, and the center G3 are not contained in the high-activity and high-selectivity region 5. That is, the molecular target C satisfies condition A, but does not satisfy condition B1. However, the arrow 7 from the center G2 to the center G3 is directed toward the high-activity and high-selectivity region 5 with increasing molecular weights, and the center G3 satisfies the activity pIC₅₀>5.0, a necessary range for synthetic expansion (condition B3). That is, the molecular target C satisfies condition A and condition B3, and can be determined as a promising drug discovery target.

### Molecular Target D

The center G2 is plotted closer to the upper left side than the center G1. However, the center G3 is not on the upper left side, but is plotted on the bottom left where the activity is low (conditions B2 and B3 are not satisfied). That is, the activity is low despite the increased molecular weight. The center G3 is also not contained in the high-activity and high-selectivity region 5 (condition B1 is not satisfied). That is, the molecular target D satisfies condition A, but does not satisfy any of the conditions B1 to B3. The molecular target D can thus be determined as a target that is undesirable as a promising drug discovery target.

### Molecular Target E

The centers G2 and G3 are plotted closer to the upper left side than the center G1. However, the center G3 is not contained in the high-activity and high-selectivity region 5 (conditions B1 and B2 are not satisfied), and does not satisfy the activity pIC₅₀>5.0, a necessary range for synthetic expansion (condition B3 is not satisfied). That is, the molecular target E satisfies condition A, but does not satisfy any of the conditions B1 to B3. The molecular target E can thus be determined as a target that is undesirable as a promising drug discovery target.

As described above, the arrow 7 for predicting the possibility of synthetic expansion can be used to determine whether a given molecular target is a promising drug discovery target. That is, by referring to the arrow 7 and the centers, a promising drug discovery target can be selected from a plurality of molecular targets.

By referring to the arrow 7 for predicting the possibility of synthetic expansion, a kinase that is promising as a drug discovery target can be automatically selected from different kinases (details will be described later). With regard to molecular target C, compounds are not present in the high-activity and high-selectivity region 5 (FIG. 6), and a quality lead compound cannot be obtained at this time. It is possible, however, to determine that the molecular target C is a promising drug discovery target from the result of determination based on the arrow 7 for molecular target C shown in FIG. 7. In other words, a prediction can be made that the molecular target C will be a molecular target that can yield a quality lead compound after screening and synthetic expansion of larger numbers of compounds (for example, several tens of thousands of compounds).

Several tens of thousands of compounds were actually screened against the molecular target C, and several tens of compounds that showed activity against the target C were evaluated for their activity against several hundred kinases, as follows. The IC₅₀ value was calculated using the inhibition rate (%) obtained according to the foregoing method, using the following formula.

IC₅₀=100×X/Y−X,

where Y is the inhibition rate (%), and X is the concentration (μM)

When the inhibition rate (%) at the maximum evaluation concentration was 20% or less, that is, when there was no activity, a fixed IC₅₀ value was used for the subsequent calculation of the entropy score used as an index of selectivity. The IC₅₀ value was 40 μM when the maximum evaluation concentration was 0.1 μM, and 400 μM when the maximum evaluation concentration was 1 μM. A fixed IC₅₀ value was also used when the inhibition rate (%) at the minimum evaluation concentration was 99% or more. In this experiment, the IC₅₀ value was 0.001 μM when the minimum evaluation concentration was 0.1 μM, and 0.01 μM when the minimum evaluation concentration was 1 μM.

The activity value (pIC₅₀), the selectivity (entropy score), and the ligand efficiency were calculated using the IC₅₀ value calculated according to the foregoing method. FIG. shows a diagram in which symbols (open square marks) representing several tens of compounds are plotted on the four-dimensional scatter diagram for target C shown in FIG. 6. A plurality of compounds was disposed in the high-activity and high-selectivity region 5. That is, the target C was shown to be a drug discovery target that can yield a high-activity and high-selectivity compound after synthetic expansion.

By referring to the arrow 7, a molecular target has a chance to be selected as a promising drug discovery target even when the symbols plotted on the four-dimensional scatter diagram showed that the molecular target is not a molecular target that can yield a quality lead compound.

### 5. Four-Dimensional Scatter Diagram Creating Device

The following describes a configuration and an operation of a four-dimensional scatter diagram creating device (an example of a visualization device) for creating and displaying the four-dimensional scatter diagram.

### 5.1 Device Configuration

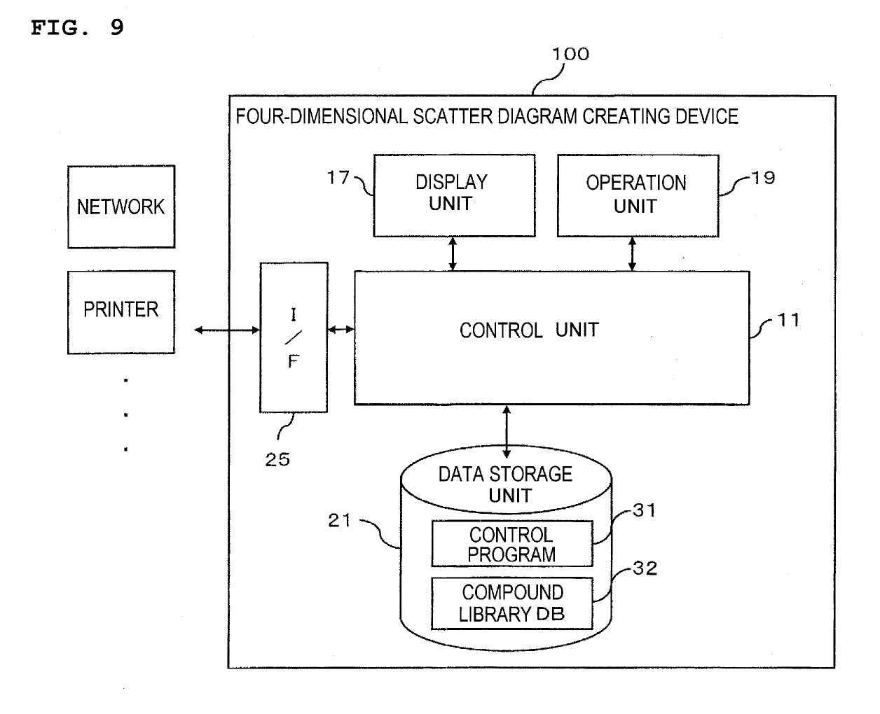

FIG. 9 is a diagram representing a hardware configuration of a four-dimensional scatter diagram creating device that creates and displays the four-dimensional scatter diagram. The four-dimensional scatter diagram creating device 100 is realized by an information processing device such as a personal computer. The four-dimensional scatter diagram creating device 100 includes a control unit 11 for controlling the overall operation, a display unit 17 for displaying information on a screen, an operation unit 19 to be operated by a user, and a data storage unit 21 for storing data and programs.

The display unit 17 is realized by, for example, a liquid crystal display device or an organic EL display device. The operation unit 19 includes a keyboard, a mouse, a touch panel, and/or so on.

The four-dimensional scatter diagram creating device 100 further includes an interface unit 25 for connecting the device 100 to external devices and a network. The interface unit 25 is connectable to a wide range of devices that conforms to USE, HDMI®, and other interface standards (including, for example, printers, communication devices, and input devices), and enables communications of data and control commands between the connected device and the four-dimensional scatter diagram creating device 100.

The control unit 11 controls the overall operation of the four-dimensional scatter diagram creating device 100, and is realized by a CPU or an MPU that executes a program to enable predetermined functions. The program executed by the control unit 11 may be provided via a communication line, or a recording medium such as a CD, a DVD, and a memory card. The control unit 11 may be realized by a dedicated hardware circuit (e.g., FPGA, ASIC) designed to enable predetermined functions.

The data storage unit 21 is a device for storing data and programs, and may be realized by, for example, a hard disc (HDD), an SSD, a semiconductor memory device, and/or an optical disk. The data storage unit 21 stores a control program 31 for creating and displaying a four-dimensional scatter diagram, a compound library database (hereinafter, referred to as “compound library DB”) 32 for storing compound data, and information of created four-dimensional scatter diagrams.

The compound library DB 32 is a database that manages information concerning features of each of a plurality of compounds. Specifically, the compound library DB 32 stores at least feature values concerning the activity and the selectivity against a plurality of kinases, the molecular weight of compounds, and the ligand efficiency of compounds, for each compound. The compound library DB 32 has, for example, the following format.

TABLE 1
Compound name

Name of kinase of interest
Activity value of compound against kinase of interest
Selectivity of compound against kinase of interest
Molecular weight of compound
Ligand efficiency of compound
. . .

That is, the compound library DB 32 stores feature values concerning the activity and the selectivity against a plurality of kinases, the molecular weight of compounds, and the ligand efficiency of compounds, for each of a plurality of compounds. The compound library DB 32 may be provided by a recording medium such as a CD, a DVD, and a memory card, or by an external server via a communication line.

### 5.2 Device Operation

### 5.2.1 Display of Four-Dimensional Scatter Diagram

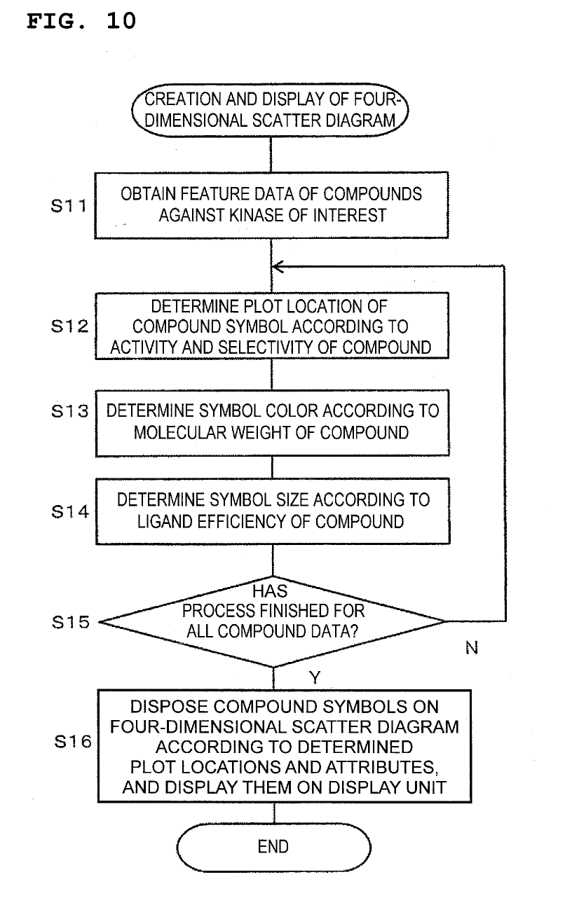

The operation of the four-dimensional scatter diagram creating device 100 is described below. FIG. 10 is a flowchart representing an operation of displaying the four-dimensional scatter diagram by the four-dimensional scatter diagram creating device 100. The display operation of the four-dimensional scatter diagram by the four-dimensional scatter diagram creating device 100 is described with reference to FIG. 10.

The control unit 11 obtains information concerning feature values of various compounds against a molecular target of interest for extraction of a lead compound from the compound library DB 32 (S11). Specifically, the control unit 11 obtains, from the compound library DB 32, at least information concerning the activity and the selectivity against the molecular target, the molecular weight, and the ligand efficiency, for each compound. Here, the control unit 11 may select and obtain information only for compounds that satisfy predetermined conditions (for example, an inhibition rate of 20% or more at the maximum evaluation concentration) in the compounds contained in the compound library DB 32.

For one of the obtained compounds, the control unit 11 determines a location of the symbol representing the compound to be plotted on a four-dimensional scatter diagram, using the activity and the selectivity of the compound against the molecular target (S12).

The control unit 11 also determines a color of the symbol representing the compound, using the molecular weight of the compound (S13). Specifically, the control unit 11 sets the color of the symbol to red for the symbol when the molecular weight is less than 300, to yellow when the molecular weight is 300 or more and less than 350, and to blue when the molecular weight is 350 or more.

The control unit 11 then determines the size of the symbol representing the compound, using the ligand efficiency of the compound (S14). Specifically, the control unit 11 sets a symbol size according to the ligand efficiency value. To be more specific, the control unit 11 sets larger symbol size as the ligand efficiency value becomes larger, and smaller symbol size as the ligand efficiency value becomes smaller. The symbols may be represented with a constant size when the ligand efficiency values are larger than a certain value, and with a constant size when the ligand efficiency values are smaller than a certain value.

The location and the attributes (color and size) of a symbol are determined for a compound in the manner described above (S12 to S14). Subsequently, the control unit 11 determines the location and the attributes (color and size) of the symbol to be disposed on a four-dimensional scatter diagram for the rest of the compounds obtained from the compound library DB 32 (S15).

Upon determining the location and the attributes (color and size) of the symbol to be disposed on a four-dimensional scatter diagram for all of the obtained compounds (YES in S15), the control unit 11 disposes the compound symbols on a selectivity-activity two-dimensional plane on the basis of the locations and the attributes (color and size) determined for the symbols, and creates a four-dimensional scatter diagram (i.e., image data representing a four-dimensional scatter diagram), and displays it on the display unit 17 (S16). As a result, the four-dimensional scatter diagram, for example, as shown in FIG. 1, is displayed on the display unit 17. Here, the control unit 11 may store image data representing the four-dimensional scatter diagram in the data storage unit 21, or may output the image data to an external device via the interface unit 25, in addition to or instead of displaying the generated four-dimensional scatter diagram on the display unit 17.

The control unit 11 also displays a box representing the high-activity and high-selectivity region 5 on the four-dimensional scatter diagram. The high-activity and high-selectivity region 5 is a region containing compounds that are more desirable as lead compounds, and where, for example, the activity (pIC₅₀) >8.0, and the selectivity (entropy score) <2.0, or where the activity (pIC₅₀) >7.0, and the selectivity (entropy score)<3.0.

The control unit 11 may be adapted to extract a compound contained in the high-activity and high-selectivity region 5 as a candidate lead compound, and store information concerning the extracted compound (e.g., compound name) in the data storage unit 21 by associating it with the molecular target, or display the information concerning the extracted compound on the display unit 17. The control unit 11 also may be adapted to extract only a compound having a molecular weight and/or a ligand efficiency satisfying the predetermined conditions from the compounds contained in the high-activity and high-selectivity region 5. A compound that is more desirable as a lead compound can be easily recognized by referring to the information concerning the compound stored in the data storage unit 21 or displayed on the display unit 17.

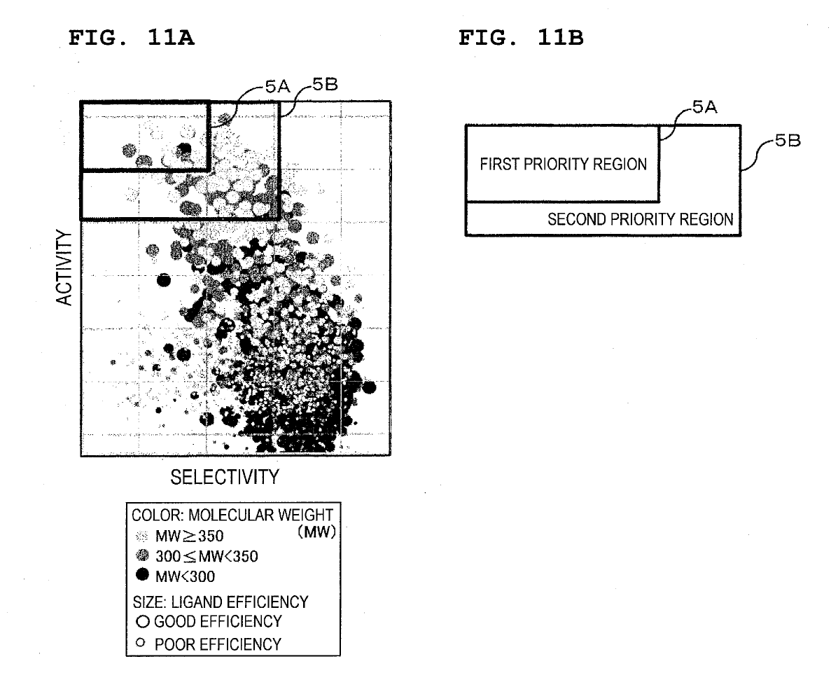

In the high-activity and high-selectivity region, the control unit 11 may display a box indicative of a region (second priority region) containing promising compounds 5B, and a box indicative of a region (first priority region) containing more promising compounds 5A, as shown in FIGS. 11A and 11B. For example, the first priority region 5A is set to a region where the activity (pIC₅₀) is 8 or more, and the selectivity (entropy score) is 2 or less. The second priority region 5B is set to a region where the activity (pIC₅₀) is 7 or more and less than 8, and the selectivity (entropy score) is more than 2 and 3 or less. In this way, a candidate lead compound to be extracted can be recognized stepwise from higher to lower priorities.

The flowchart shown in FIG. 10 describes the four-dimensional scatter diagram displaying a process for a single molecular target. When a plurality of four-dimensional scatter diagrams needs to be displayed for plural molecular target at the same time, for example, as shown in FIGS. 3 and 6, the process of the flowchart shown in FIG. 10 may be performed for each molecular target.

### 5.2.2 Arrow Indicator for Prediction of Possibility of Synthetic Expansion

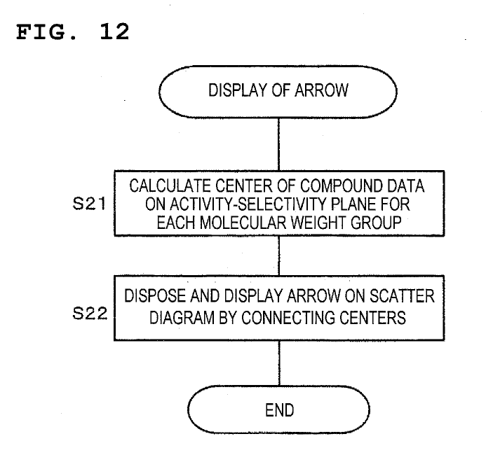

FIG. 12 is a flowchart representing a process for generating the arrow 7 for predicting possibility of synthetic expansion, as shown in FIGS. 4A-4B and 5A-5B and elsewhere. With reference to FIG. 12, the process for generating the arrow 7 for predicting possibility of synthetic expansion in the four-dimensional scatter diagram creating device 100.

The control unit 11 manages the compounds that are divided into three groups by molecular weight, specifically a first group with a molecular weight of less than 300, a second group with a molecular weight of 300 or more and less than 350, and a third group with a molecular weight of 350 or more. For these molecular weight groups, the control unit 11 calculates the centers G1, G2, and G3 of distributions of symbols on the selectivity-activity two-dimensional plane (distributions on the selectivity-activity two-dimensional plane) (S21).

Specifically, for the compounds belonging to the first group, the control unit 11 calculates the mean values of activity and selectivity using the formula (1) to obtain the center G1 of the distribution of the compounds belonging to the first group. In the same fashion, the control unit 11 obtains the center G2 of the distribution of the compounds belonging to the second group by calculating the mean values of activity and selectivity for the compounds belonging to the second group, using the formula (1). For the compounds belonging to the third group, the control unit 11 calculates the mean values of activity and selectivity, using the formula (1) to obtain the center G3 of the distribution of the compounds belonging to the third group. The centers G1, G2, and G3 may be calculated using the weighted formula (3).

The control unit 11 connects centers G1 and G2, and centers G2 and G3 of groups having the adjacent molecular weight ranges, and displays the result on the four-dimensional scatter diagram (S22). As a result, the arrows 7 representing a distribution change are displayed on the four-dimensional scatter diagram, for example, as shown in FIGS. 4A and 4B.

The control unit 11 may display the arrows 7 by themselves, without the plotted symbols shown in FIGS. 5A and 5B. Arrows for a plurality of molecular targets may be displayed side by side as shown in FIG. 7. In this case, the process of the flowchart shown in FIG. 12 is executed for each molecular target.

The control unit 11 may be adapted to determine whether the molecular target is a promising drug discovery target, according to the locations of the calculated centers G1 to G3, and the direction (slope) of the arrow 7, and store the result of determination in the data storage unit 21, or display the result in the display unit 17. In this way, it can be presented to the user of the device whether the molecular target represented in the four-dimensional scatter diagram is a promising drug discovery target.

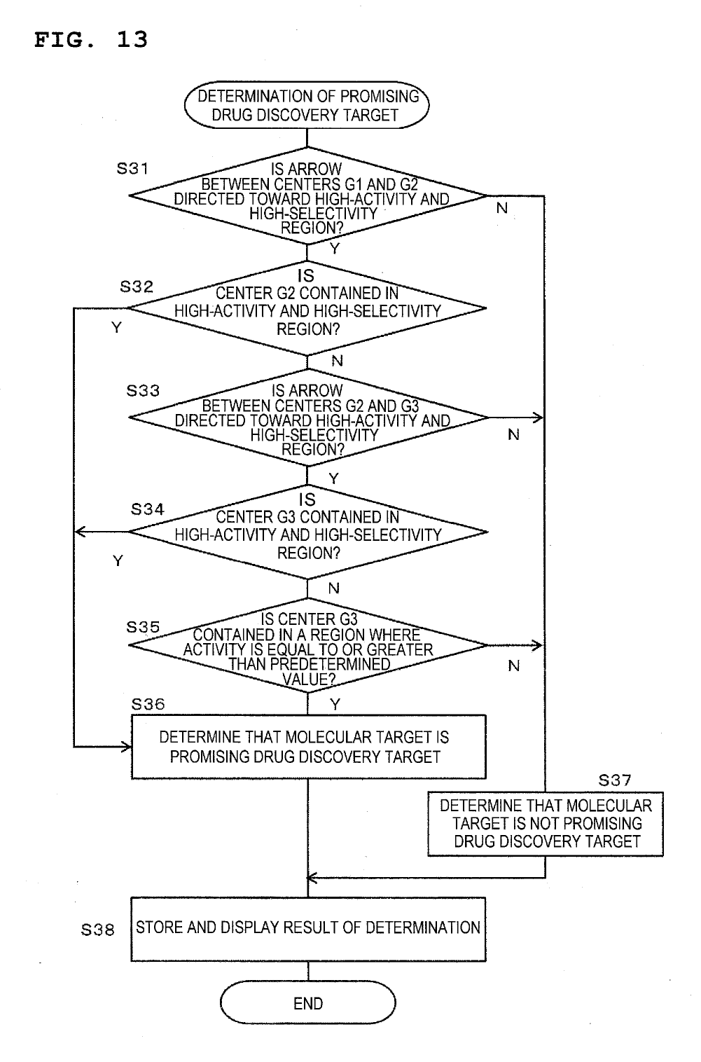

The following describes an operation for determining whether the molecular target is a promising drug discovery target according to the locations of the centers and the direction of the arrow. FIG. 13 is a flowchart showing the procedure performed by the control unit 11.

First, the control unit 11 determines whether the arrow between the centers G1 and G2 (the arrow from center G1 to center G2) is directed toward the high-activity and high-selectivity region 5 (condition A) (S31). Specifically, the control unit 11 determines whether the arrow between the centers G1 and G2 is directed toward the upper left side of the selectivity-activity two-dimensional plane. When the arrow between the centers G1 and G2 is not directed toward the high-activity and high-selectivity region 5 (NO in S31), the control unit 11 determines that the molecular target is not a promising drug discovery target (S37).

When the arrow between the centers G1 and G2 is directed toward the high-activity and high-selectivity region 5 (YES in S31), the control unit 11 determines whether the center G2 is contained in the high-activity and high-selectivity region 5 (condition B1) (S32). When the center G2 is contained in the high-activity and high-selectivity region 5 (YES in S32), the control unit 11 determines that the molecular target is a promising drug discovery target (S36).

When the center G2 is not contained in the high-activity and high-selectivity region 5 (NO in S32), the control unit 11 determines whether the arrow between the centers G2 and G3 (the arrow from center G2 to center G3) is directed toward the high-activity and high-selectivity region 5 (S33). When the arrow between the centers G2 and G3 is not directed toward the high-activity and high-selectivity region 5 (NO in S33), the control unit 11 determines that the molecular target is not a promising drug discovery target (S37). When the arrow between the centers G2 and G3 is directed toward the high-activity and high-selectivity region 5 (YES in S33), the control unit 11 determines whether the center G3 is contained in the high-activity and high-selectivity region 5 (condition B2) (S34). When the center G3 is contained in the high-activity and high-selectivity region 5 (YES in S34), the control unit 11 determines that the molecular target is a promising drug discovery target (S36).

When the center G3 is not contained in the high-activity and high-selectivity region 5 (NO in S34), the control unit 11 determines whether the center G3 is contained in a region where the activity value is equal to or greater than a predetermined value (for example, pIC₅₀ is 5 or more) (condition B3) (S35). When the center G3 is contained in the region where the activity value is equal to or greater than the predetermined value (YES in S35), the control unit 11 determines that the molecular target is a promising drug discovery target (S36). When the center G3 is not contained in the region where the activity value is equal to or greater than the predetermined value (NO in S35), the control unit 11 determines that the molecular target is not a promising drug discovery target (S37).

In this manner, the control unit 11 determines whether the molecular target is a promising drug discovery target according to the locations of the centers and the direction of the arrow, and stores the result of determination in the data storage unit 21, or displays the result on the display unit 17 (S38).

The high-activity and high-selectivity region 5 is a preferred region for locating the center therein. For example, the high-activity and high-selectivity region 5 may be set as a region where the activity (pIC₅₀) >5.0 and the selectivity (entropy score)<4.0, a region where the activity (pIC₅₀) >6.0 and the selectivity (entropy score)<3.0, a region where the activity (pIC₅₀) >7.0 and the selectivity (entropy score) <2.5, or a region where the activity (pIC₅₀) >7.0 and the selectivity (entropy score)<2.0.

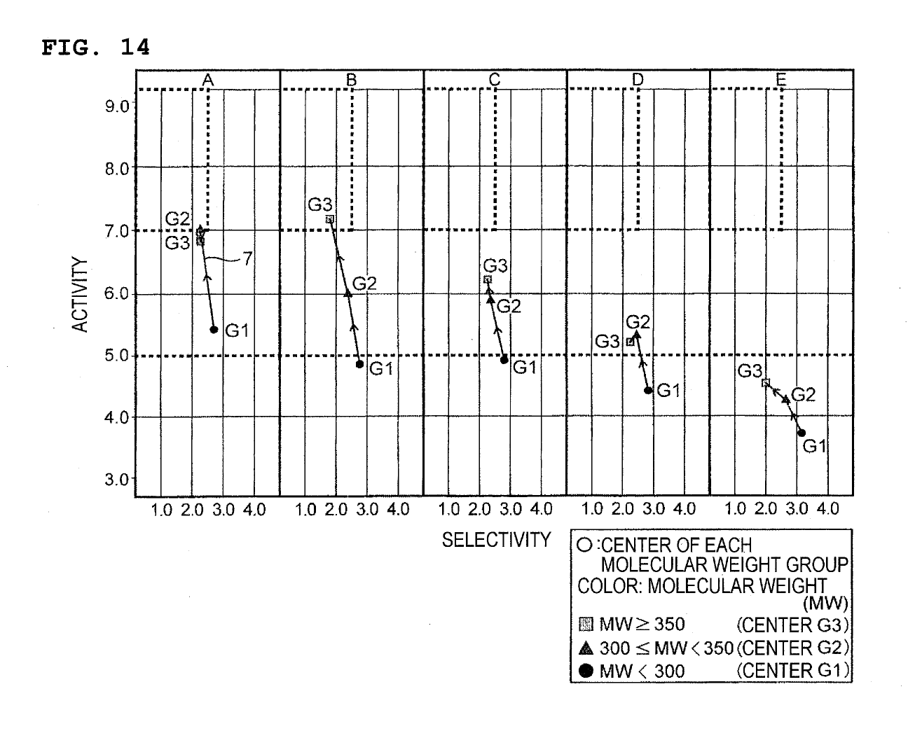

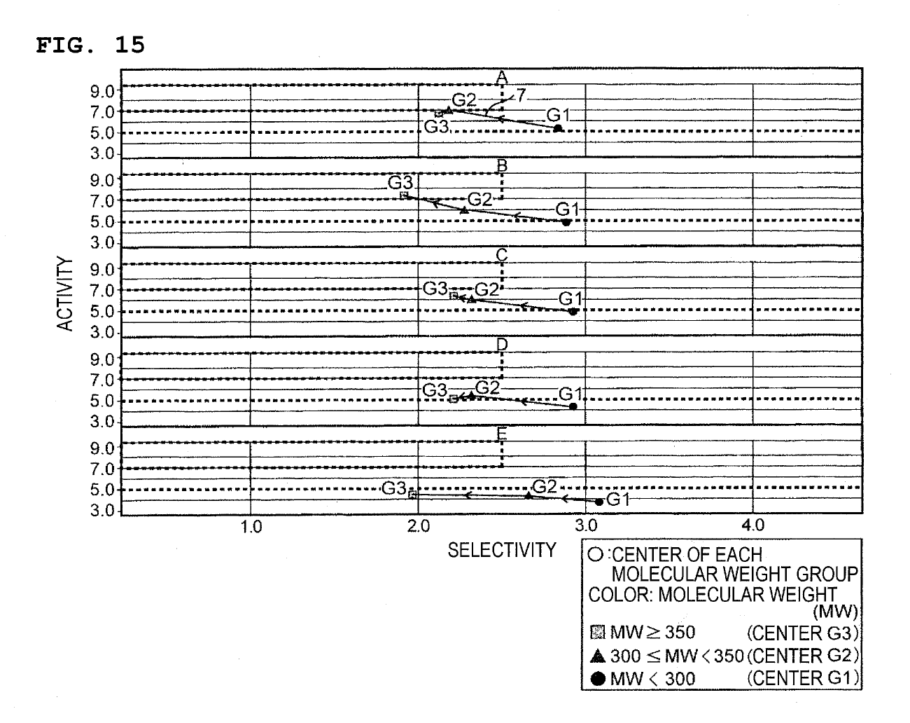

The method of displaying the arrows for predicting the possibility of synthetic expansion for a plurality of molecular targets is not limited to one as shown in FIG. 7 in which the allows are arranged vertically and horizontally. For example, the arrows may be displayed, arranged either horizontally as shown in FIG. 14, or vertically as shown in FIG. 15. Both cases can enable grasping the patterns of arrows for each molecular target, and determining whether the molecular target is a promising drug discovery target according to the location and the direction of the arrow.

### 6. Effects, and Other

In the four-dimensional scatter diagram described above, the location of a symbol to be disposed is determined according to the selectivity (an example of the first feature), and the activity value (second feature) of a compound against a molecular target, and the attributes (color, size) of the symbol are determined according to the molecular weight (an example of the third feature) and the ligand efficiency (example of the fourth feature) of the compound. The four-dimensional scatter diagram enables grasping data in a comprehensive fashion, and predicting the possibility of synthetic expansion. With the four-dimensional scatter diagram, it is also possible to understand the molecular weight distribution, an important factor of a quality lead compound, and to recognize the ligand efficiency in one glance. A compound that is more desirable as a lead compound also can be easily recognized by focusing on the predetermined region (high-activity and high-selectivity region 5) of the four-dimensional scatter diagram.

In the lead compound extraction method disclosed in the present embodiment, a lead compound is extracted from compounds represented by symbols disposed in the predetermined region (high-activity and high-selectivity region) 5 of the four-dimensional scatter diagram. In this way, the method enables extracting a quality lead compound having good potential for synthetic expansion.

An arrow representing a change in the distribution of symbols in a group of compounds divided by molecular weight may be displayed on the four-dimensional scatter diagram. In the drug discovery target selecting method disclosed in the present embodiment, whether to select a predetermined target as a drug discovery target for drug discovery is determined according to the direction of change of the distribution of symbols in a group of compounds divided by molecular weight on the four-dimensional scatter diagram. By determining whether the target is a drug discovery target according to a change in the distribution of symbols in a group of compounds divided by molecular weight, it is possible to determine whether a drug candidate compound would be created against the drug discovery target of interest in the future after synthetic expansion.

The foregoing embodiment provides the four-dimensional scatter diagram creating device 100 that creates the four-dimensional scatter diagram representing the features of a plurality of compounds against a predetermined. drug discovery target and/or molecular target. The four-dimensional scatter diagram creating device 100 includes the control unit 11. The control unit 11 functions as a unit for obtaining feature information concerning several features of each of a plurality of compounds (S11), and as scatter diagram creating unit for creating and outputting a four-dimensional scatter diagram in which symbols each representing each compound are disposed according to the obtained feature information for the plurality of compounds (S12 to S16). such a four-dimensional scatter diagram creating device 100 can create the four-dimensional scatter diagram.

### Other Embodiments

The embodiment described above discloses an exemplary implementation of the present invention, and is not intended to limit the ideas of the present invention. Various changes, modifications, replacements, additions, and omissions may be made to the techniques disclosed. The following describes some of such variations.

(1) The foregoing description was given through the case where the features of the compounds plotted on the four-dimensional scatter diagram are activity (an example of the first feature), selectivity (an example of the second feature), molecular weight (an example of the third feature), and ligand efficiency (an example of the fourth feature). However, the compound features are not limited to these. The compound features may be evaluation items used for drug discovery, including, for example, activity, selectivity, molecular weight, ligand efficiency, lipid solubility (e.g., log P, log D, c log P, A log P, and M log P), number of heavy atoms, number of hydrogen bond donors, number of hydrogen bond acceptors, number of rotatable bonds, polar surface area (e.g., PSA, TPSA), number of aromatic rings, number of structural alerts, acid dissociation constant, QED (quantitative estimate of drug-likeness), CNS MPO (central nervous system multiparameter optimization), solubility, heat stability, hygrostability, photostability, membrane permeability, oral absorbability, human intestinal absorption (HIA), blood-brain barrier (BBB) transport, cytochrome P450 (e.g., CYP3A4, CYP2D6) metabolic stability, cytochrome 2450 inhibition (e.g., CYP3A4) activity, carcinogenicity, mutagenicity (e.g., Ames test), skin sensitization, accumulation, hERG inhibition, and chromosome abnormality expression. Two or more of these features may be used in combination (for example, ligand lipophilicity efficiency as a combination of activity and lipid solubility). However, the preferred combination is the combination of activity, selectivity, molecular weight, and ligand efficiency.

(2) The foregoing description was given through the case where the symbol color is set according to the molecular weight of the compound, and the symbol size is set according to the ligand efficiency. However, the symbol size may be set according to the molecular weight of the compound, and the symbol color may be set according to the ligand efficiency.

(3) The shape of the symbol was described as being circular. However, the symbol shape is not limited to this, and may be represented by any shape, including, for example, a triangle, a rectangle, a star shape, and a cross shape.

(4) Color and size were used as attributes of the symbol, and these were varied according to the compound features (molecular weight, and ligand efficiency). However, shape and three-dimensional coordinates (coordinates on the Z axis perpendicular to the plane defined by the X axis representing selectivity, and the Y axis representing activity) may additionally be used as attributes of the symbol.

Specifically, two of the attributes selected from color, size, shape, and three-dimensional coordinates may be varied according to the compound features (molecular weight, and ligand efficiency).

For example, the four-dimensional scatter diagram is three-dimensionally expressed when the Z-axis coordinates are decided according to either the molecular weight or the ligand efficiency of the compound.

(5) One of the attributes of the compound was varied according to one of the features of the compound. However, more than one attribute may be varied according to one of the features of the compound. For example, the color and shape of a symbol may be varied together according to the molecular weight of the compound.

(6) The foregoing description was given through the case where the four features of data of interest were each reflected on the location, the color, or other attributes of the symbol in the four-dimensional scatter diagram. However, the scatter diagram is not limited to this. The scatter diagram may be created by varying the attributes of the plotted symbols so that more than four features can be viewed at the same time. For example, the scatter diagram may be created by determining the location (X axis, Y axis), the color, the size, and the shape of a symbol for each of five features.

(7) The foregoing example described the data visualization method that is effective for extracting a quality lead compound or selecting a drug discovery target. However, the data visualization method using the four-dimensional scatter diagram disclosed in the foregoing embodiment is not limited to visualization of feature data of candidate compounds used for the extraction of a lead compound or the selection of a drug discovery target. The data visualization method disclosed in the foregoing embodiment is also applicable to a visualization method used to visualize ordinary data having four- or higher-dimensional features. Such a visualization method can be effectively applied for the analysis of big data, and for deciding the course of action based on the result of such an analysis.

For example, the data visualization method is applicable to visualize a wide range of data in the following areas.
Medicine (for example, medical data analysis, dosing information analysis, test result analysis, vital data analysis, disease risk analysis, infection prediction analysis, community information analysis)
Finance and insurance (for example, fraud analysis, transaction analysis, risk analysis, position information analysis),
Communication and broadcasting (for example, communication log analysis, network analysis, rating analysis, content analysis)
Distribution and retail (for example, PUS data analysis, purchase log analysis, loyalty analysis, promotion analysis, call center analysis, eye-tracking analysis, repeat rate analysis, service usage analysis, point usage analysis, click stream analysis),
Manufacture (for example, quality analysis, demand analysis, traceability, failure advance detection, down time prediction)
Media, including Web (for example, access analysis, content analysis, social media analysis)
Public service and public welfare (for example, weather data analysis, earthquake data analysis, energy consumption analysis, risk analysis (e.g., defense, crime), detection of defects in bridge pier, efficient operation of social infrastructure),
Traffic (for example, automobile driving data analysis, prediction of road congestion, accident cause analysis, CO₂ emission analysis),
Tourism (for example, analysis of tourists' needs),
Farming and fishery (for example, dynamic analysis, growth analysis, prediction of fishing grounds)

Specifically, for plural pieces of data to be analyzed having at least first to fourth features, this visualization method determines the location at which a symbol representing each piece of data is to be disposed, according to the first and second features. The visualization method then determines the attributes of the symbol representing each piece of data, according to the third and fourth features. The four-dimensional scatter diagram is created by disposing each data symbol according to the location and the attributes determined above. By referring to the four-dimensional scatter diagram created in this fashion, the four features of the analyzed data can be visually recognized at the same time, and the patterns of the analyzed data can be grasped both easily and intuitively.

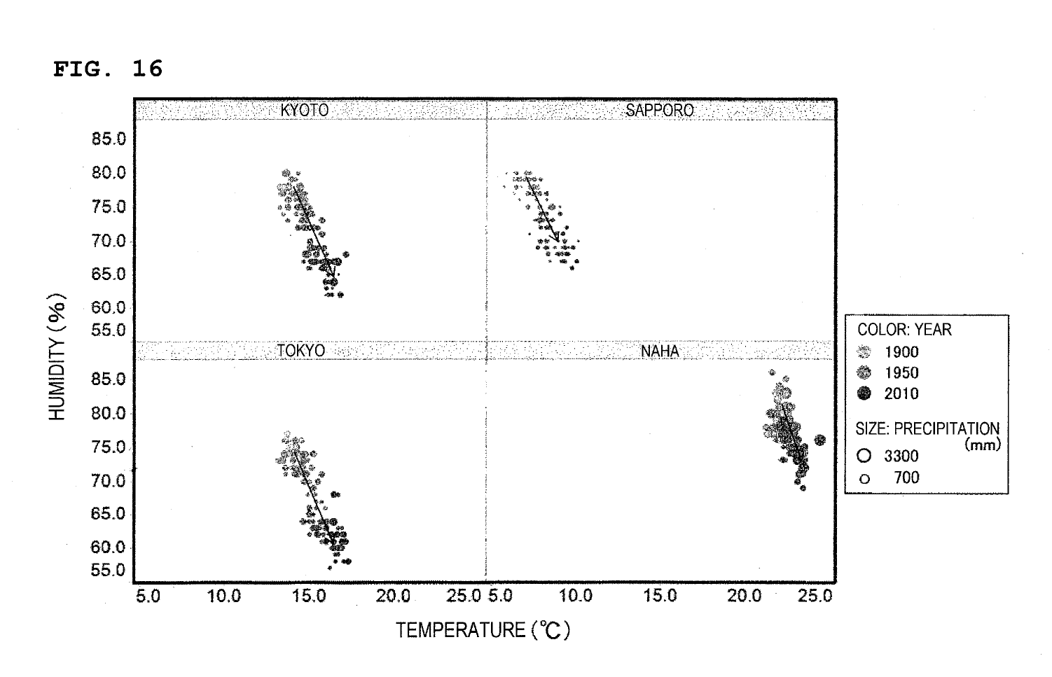

For example, the four-dimensional scatter diagram. shown in FIG. 16 may be created according to four features of weather data, specifically temperature, humidity, the year observed, and precipitation. The data were obtained from meteorological data in Japan. Specifically, the average temperature, the humidity, and the precipitation observed in Kyoto, Sapporo, Tokyo, and Okinawa from year 1900 to 2015 were used. In the four-dimensional scatter diagram, the horizontal axis represents temperature, the vertical axis represents humidity, the symbol color represents the year observed (darker colors indicate years closer to the present), and the symbol size represents precipitation. As can be seen in FIG. 16, the temperature increases from the past to the present in each city. That is, the diagram is showing global warming patterns. It is also possible to grasp a pattern for decreasing humidity levels with increasing temperatures. By referring to the four-dimensional scatter diagram for weather in this manner, changing environmental patterns can be grasped both easily and intuitively.

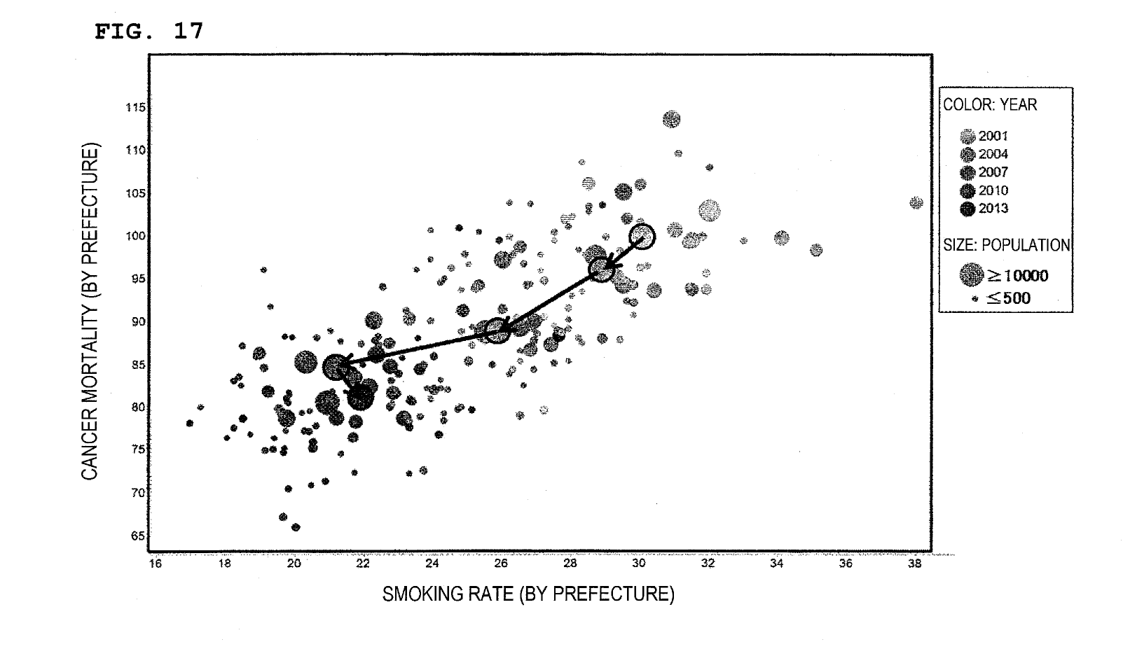

As another example, the four-dimensional scatter diagram shown in FIG. 17 can be obtained according to four features in medical data, specifically, cancer mortality, smoking rate, survey year, and population. The data were obtained from medical data in Japan. Specifically, cancer mortality by prefecture (age-adjusted mortality from malignant neoplasm for ages below 75, per 100,000 people), smoking rate by prefecture, and population data for every 3 years from year 2001 to 2013 were used. In the four-dimensional scatter diagram, the horizontal axis represents smoking rate, the vertical axis represents cancer mortality, the symbol color represents survey year (darker colors indicate years closer to the present), and the symbol size represents population. As can be seen in FIG. 17, there is a correlation between smoking rate and cancer mortality. By plotting the national average values of smoking rate and cancer mortality from each survey (thick open circles in FIG. 17), and connecting these circles with arrows, it is also possible to grasp a pattern for decreasing smoking rates and decreasing cancer mortality in almost every survey. Changing patterns of cancer mortality can be grasped both easily and intuitively by referring to the medical four-dimensional scatter diagram in this manner.

In this case, the control unit 11 of the four-dimensional scatter diagram creating device 100 may be configured to provide the following functions. Specifically, for plural pieces of analysis data having first to fourth features, the control unit 11 may determine a location of a symbol representing each piece of data according to the first and the second features. Further the control unit 11 may determine attribute of the symbol for each piece of data according to the third and the fourth features. Then the control unit 11 may create a four-dimensional scatter diagram by disposing the symbol for each piece of data according to the location and the attribute determined as above. Further the control unit may divide data into a plurality of groups under a predetermined condition with regard to the third feature, and dispose, on the scatter diagram, arrows that connect the centers of the distributions of the symbols for the data belonging to the divided groups. By referring to the direction of the arrow and the location of the center, changing patterns of the distribution of the analysis data divided for the third feature can be visually and easily recognized.

### Present Disclosure

The embodiments described above disclose the following ideas.

(1) A method for extracting a lead compound from a plurality of compounds against a drug discovery target.

The method includes the steps of:

creating a scatter diagram for a plurality of compounds by disposing symbols representing the respective compounds according to a plurality of features of the respective compounds; and

extracting a lead compound from the compounds represented by the symbols disposed in a predetermined region of the scatter diagram.

A location of the symbol to be disposed on the scatter diagram is determined according to first and second features of the compound, and attributes of the symbol on the scatter diagram are determined according to third and fourth features of the compound.

(2) In the method of (1), the attributes of the symbol may include at least two selected from a color, a shape, and a size concerning the symbols, and three-dimensional coordinates representing a location in a direction perpendicular to a plane on which the symbols are disposed according to the first and second features.

(3) In the method of (1), the first feature may be selectivity of the compounds against the predetermined drug discovery target, the second feature may be activity of the compound against the predetermined drug discovery target, the third feature may be a molecular weight of the compound, and the fourth feature may be a ligand efficiency of the compound.

(4) In the method of (3), the predetermined region may be a region in which the selectivity and the activity of the compound are equal to or greater than respective predetermined values.

(5) In the method of (4), a compound having a ligand efficiency of 0.3 or more may be extracted from the compounds represented by the symbols disposed in the predetermined region.

(6) In the method of any of (1) to (5), the drug discovery target may be an enzyme, a receptor, or a transporter protein.

(7) A method for extracting a lead compound from a plurality of compounds against a drug discovery target.

The method includes the steps of:

creating a scatter diagram for a plurality of compounds by disposing symbols representing the respective compounds according to a plurality of features of the respective compounds; and

extracting a lead compound from the compounds represented by the symbols disposed in a predetermined region of the scatter diagram.

Locations of the symbols to be disposed on the scatter diagram are determined according to first and second features of the respective compound. The first feature is selectivity of the compound against the predetermined drug discovery target. The second feature is activity of the compound against the predetermined drug discovery target. The predetermined region is a region in which the selectivity and the activity are equal to or greater than respective predetermined values. A compound having a ligand efficiency of 0.3 or more is extracted from the compounds represented by the symbols disposed in the predetermined region.

(8) A method for selecting a drug discovery target.

The method includes the steps of:

creating a scatter diagram for a plurality of compounds against a predetermined molecular target by disposing symbols representing the respective compounds according to a plurality of features of the respective compounds; and

selecting the predetermined molecular target as a drug discovery target according to a distribution of the symbols disposed on the scatter diagram.

A location of the symbol to be disposed on the scatter diagram is determined according to first and second features of the compounds, and attributes of the symbol on the scatter diagram are determined according to third and fourth features of the compound. The compounds are divided into a plurality of groups under a predetermined condition regarding the third feature. In the selecting step, it is determined whether to select the predetermined molecular target as a drug discovery target according to the direction of change in the distributions of the symbols of the compounds belonging to the respective groups.

(9) In the method of (8), the attributes of the symbol may include at least two selected from a color, a shape, and a size concerning the symbols, and three-dimensional coordinates representing a location in a direction perpendicular to a plane on which the symbols are disposed according to the first and second features.

(10) In the method of (8), the first feature is selectivity of the compound against the predetermined molecular target, the second feature is activity of the compound against the predetermined molecular target, the third feature is a molecular weight of the compound, and the fourth feature is a ligand efficiency of the compound.

(11) In the method of (10), the compounds may be divided into a plurality of groups according to the molecular weight, and an arrow connecting the centers of the distributions of the symbols of the compounds belonging to the respective groups may be disposed on the scatter diagram.

(12) In the method of (11), the molecular target may be selected as a drug discovery target, when the arrow connecting the centers of the distributions of the symbols of the compounds belonging to the respective groups is directed toward a predetermined region of the scatter diagram.

(13) In the method of (12), the molecular target may be selected as a drug discovery target, when the location of the center of the distribution representing an end point of change on the scatter diagram is in a region in which the selectivity is equal to or greater than a predetermined value, and in which the activity is equal to or greater than a predetermined value.

(14) In the method of any one of (8) to (13), the drug discovery target, and/or the molecular target may be an enzyme, a receptor, or a transporter protein.

(15) A scatter diagram creating device for creating a scatter diagram that represents features of a plurality of compounds against a predetermined drug discovery target.

The device includes:

an obtaining unit for obtaining feature information regarding various features of the compounds, for a plurality of compounds; and

a scatter diagram creating unit for creating a scatter diagram for the plurality of compounds, by disposing symbols representing the compounds according to the obtained feature information, and outputting the scatter diagram.

The scatter diagram creating unit determines the locations of the symbols to be disposed on the scatter diagram, according to first and second features of the respective compounds, determines attributes of the symbols according to third and fourth features of the respective compounds, and disposes the symbols representing the compounds on the scatter diagram according to the determined locations and the determined attributes.

(16) In the device of (15), the attributes of the symbols may include at least two selected from a color, a shape, and a size concerning the symbols, and three-dimensional coordinates representing a location in a direction perpendicular to a plane on which the symbols are disposed according to the first and second features.

(17) In the device of (15), the first feature may be selectivity of the compound against the predetermined drug discovery target, the second feature may be activity of the compound against the predetermined drug discovery target, the third feature may be a molecular weight of the compound, and the fourth feature may be a ligand efficiency of the compound.

(18) In the device of (17), the scatter diagram. creating unit may dispose, on the scatter diagram, information representing a region in which the selectivity of the compound is equal to or greater than a predetermined value and the activity of the compound is equal to or greater than a predetermined value.

(19) The device of (18) may further include an extracting unit for extracting, as a lead compound, at least one of the compounds represented by the symbols disposed in the region.

(20) In the device of (17), the scatter diagram creating unit may divide a plurality of compounds into a plurality of groups according to the molecular weight, and may dispose on the scatter diagram an arrow connecting the centers of distributions of the symbols of the compounds belonging to the respective groups.

(21) In the device of any one of (15) to (20), the drug discovery target may be an enzyme, a receptor, or a transporter protein.

(22) A program for controlling a computer to create a scatter diagram that represents features of a plurality of compounds against a predetermined drug discovery target.

The program causes the computer to operate as:

an obtaining unit for obtaining feature information regarding various features of the compound, for a plurality of compounds; and

a scatter diagram creating unit for creating a scatter diagram for the plurality of compounds, by disposing symbols representing the compounds according to the obtained feature information.

The scatter diagram creating unit determines, for the respective compounds, the locations of the symbols to be disposed on the scatter diagram according to first and second features of the respective compounds, determines attributes of the symbols according to third and fourth features of the respective compounds, and disposes the symbols representing the compounds on the scatter diagram according to the determined locations and the determined attributes.

(23) A first method for visualizing a pattern of a plurality of pieces of data having at least first to fourth features,

The method includes:

determining a location on which a symbol representing each piece of data is to be disposed, according to the first and second features;

determining attributes of the symbol representing each piece of data, according to the third and fourth features; and

disposing the symbol representing each piece of data on a scatter diagram according to the determined location and the determined attributes.

(24) In the method of (23), the plurality of pieces of data may be divided into groups under a predetermined condition regarding the third feature. An arrow connecting the centers of distributions of the symbols of the data belonging to the groups may be disposed on the scatter diagram.

(25) A second method for visualizing a pattern of a plurality of data having at least a first to a third feature.

The method includes:

determining a location on which a symbol representing each piece of data is disposed, according to the first and second features; and

disposing the symbol representing each piece of data on a scatter diagram according to the determined location;

dividing the plurality of pieces of data into a plurality of groups under a predetermined condition regarding the third feature; and

disposing an arrow connecting centers of distributions of the symbols of the data belonging to the respective groups on the scatter diagram.

(26) A device for visualizing a pattern of a plurality of pieces of data having at least first to fourth features.

The device includes:

an obtaining unit for obtaining feature information regarding features of the data, for the respective pieces of data; and

a scatter diagram creating unit for creating a scatter diagram according to the feature information obtained for the data.

The scatter diagram creating unit determines the location on which a symbol representing each piece of data is disposed, according to the first and second features, determines attributes of the symbol representing each piece of data according to the third and fourth features, and disposes on the scatter diagram the symbol representing each piece of data according to the determined location and the determined attributes.

While the present invention has described with certain embodiments of the invention as specific examples of the invention, it will be apparent to a skilled person that various variations, modifications, substitutions, additions, and omissions may be made thereto within the scope of the claims and the equivalence thereof.

1. A method for extracting a lead compound from a plurality of compounds against a drug discovery target, the method comprising the steps of:
creating a scatter diagram for a plurality of compounds by disposing symbols representing the respective compounds according to a plurality of features of the respective compounds; and
extracting a lead compound from the compounds represented by the symbols disposed in a predetermined region of the scatter diagram,
wherein a location of the symbol to be disposed on the scatter diagram is determined according to first and second features of the compound, and attributes of the symbol are determined according to third and fourth features of the compound.

2. The method according to claim 1, wherein the attributes of the symbol include at least two selected from a color, a shape, and a size concerning the symbols, and three-dimensional coordinates representing a location in a direction perpendicular to a plane on which the symbols are disposed according to the first and second features.

3. The method according to claim 1, wherein the first feature is selectivity of the compound against the predetermined drug discovery target, the second feature is activity of the compound against the predetermined drug discovery target, the third feature is a molecular weight of the compound, and the fourth feature is a ligand efficiency of the compound.

4. The method according to claim 3, wherein the predetermined region is a region in which the selectivity and the activity of the compound are equal to or greater than respective predetermined values.

5. The method according to claim 4, wherein a compound having a ligand efficiency of 0.3 or more is extracted from the compounds represented by the symbols disposed in the predetermined region.

6. (canceled)

7. (canceled)

8. A method for selecting a drug discovery target, comprising the steps of:
creating a scatter diagram for a plurality of compounds against a predetermined molecular target, by disposing symbols representing the respective compounds according to a plurality of features of the respective compounds; and
selecting the predetermined molecular target as a drug discovery target according to a distribution of the symbols disposed on the scatter diagram,
wherein a location of the symbol to be disposed on the scatter diagram is determined according to first and second features of the compound, and attributes of the symbol are determined according to third and fourth features of the compounds,
wherein the compounds are divided into a plurality of groups under a predetermined condition regarding the third feature, and
wherein in the selecting step, it is determined whether to select the predetermined molecular target as a drug discovery target, according to a direction and an end point of change in the distributions of the symbols of the compounds belonging to the respective groups.

9. The method according to claim 8, wherein the attributes of the symbol include at least two selected from a color, a shape, and a size concerning the symbols, and three-dimensional coordinates representing a location in a direction perpendicular to a plane on which the symbols are disposed according to the first and second features.

10. The method according to claim 8, wherein the first feature is selectivity of the compound against the predetermined molecular target, the second feature is activity of the compound against the predetermined molecular target, the third feature is a molecular weight of the compound, and the fourth feature is a ligand efficiency of the compound.

11. The method according to claim 10, wherein
the compounds are divided into a plurality of groups according to the molecular weight, and
an arrow connecting the centers of the distributions of the symbols of the compounds belonging to the respective groups is disposed on the scatter diagram.

12. The method according to claim 11, wherein the molecular target is selected as a drug discovery target, when the arrow connecting the centers of the distributions of the symbols of the compounds belonging to the respective groups is directed toward a predetermined region of the scatter diagram.

13. The method according to claim 12, wherein the molecular target is selected as a drug discovery target, when the location of the center of the distribution representing an end point of change on the scatter diagram is in a region in which the selectivity is equal to or greater than a predetermined value, and in which the activity is equal to or greater than a predetermined value.

14. (canceled)

15. A scatter diagram creating device for creating a scatter diagram that represents features of a plurality of compounds against a predetermined drug discovery target, the device comprising:
an obtaining unit for obtaining feature information regarding various features of the compound, for a plurality of compounds; and
a scatter diagram creating unit for creating a scatter diagram for the plurality of compounds, by disposing symbols representing the compounds according to the obtained feature information, and outputting the scatter diagram,
wherein the scatter diagram creating unit
determines the locations of the symbols to be disposed on the scatter diagram according to first and second features of the respective compounds,
determines attributes of the symbols according to third and fourth features of the respective compounds, and
disposes the symbols representing the compounds on the scatter diagram according to the determined locations and the determined attributes.

16. The device according to claim 15, wherein the attributes of the symbols include at least two selected from a color, a shape, and a size concerning the symbols, and three-dimensional coordinates representing a location in a direction perpendicular to a plane on which the symbols are disposed according to the first and second features.

17. The device according to claim 15, wherein the first feature is selectivity of the compound against the predetermined drug discovery target, the second feature is activity of the compound against the predetermined drug discovery target, the third feature is a molecular weight of the compound, and the fourth feature is a ligand efficiency of the compound.

18. The device according to claim 17, wherein the scatter diagram creating unit disposes, on the scatter diagram, information representing a region in which the selectivity of the compound is equal to or greater than a predetermined value and the activity of the compound is equal to or greater than a predetermined value.

19. The device according to claim 18, further comprising an extracting unit for extracting, as a lead compound, at least one of the compounds having the symbols disposed in the region.

20. The device according to claim 17, wherein the scatter diagram creating unit divides the plurality of compounds into a plurality of groups according to the molecular weight, and disposes, on the scatter diagram, an arrow connecting the centers of distributions of the symbols of the compounds belonging to the respective groups.

21. (canceled)

22. (canceled)

23. A method for visualizing a pattern of a plurality of pieces of data having at least first to fourth features, the method comprising:
determining a location on which a symbol representing each piece of data is to be disposed, according to the first and second features;
determining attributes of the symbol representing each piece of data, according to the third and fourth features; and
disposing the symbol representing each piece of data on a scatter diagram according to the determined location and the determined attributes.

24. The method according to claim 23, wherein
the plurality of pieces of data are divided into groups under a predetermined condition regarding the third feature, and
an arrow connecting the centers of distributions of the symbols of the data belonging to the groups is disposed on the scatter diagram.

25. (canceled)

26. A device for visualizing a pattern of a plurality of pieces of data having at least first to fourth features, the device comprising:
an obtaining unit for obtaining feature information regarding features of data, for each piece of data; and
a scatter diagram creating unit for creating a scatter diagram according to the feature information obtained for the data,
wherein the scatter diagram creating unit
determines the location on which a symbol representing each piece of data is disposed, according to the first and second features,
determines attributes of the symbol representing each piece of data, according to the third and fourth features, and
disposes, on the scatter diagram, the symbol representing each piece of data according to the determined location and the determined attributes.
